(19) United States
(12) Patent Application Publication
(10) Pub. No.: US 2025/0122526 A1
(43) Pub. Date: Apr. 17, 2025

(54) CHIMERIC REPRESSORS AND METHODS OF USING THE SAME

(71) Applicant: SAGITTARIUS BIO, INC, Thousand Oaks, CA (US)

(72) Inventors: Kayvan NIAZI, Thousand Oaks, CA (US); Shahrooz RABIZADEH, Thousand Oaks, CA (US)

(21) Appl. No.: 18/723,190

(22) PCT Filed: Dec. 22, 2022
(86) PCT No.: PCT/US2022/082242

§ 371 Date: Jun. 21, 2024

Related U.S. Application Data
(60) Provisional application No. 63/292,956, filed on Dec. 22, 2021.

(51) Int. Cl. C12N 15/85; C12N 15/63

(52) U.S. Cl. CPC C12N 15/85; C12N 15/635; C12N 2840/002

(57) ABSTRACT

Embodiments provided for herein relate to a CRO repressor system, compositions comprising the same, and methods of using the same. In some embodiments, the repressor system comprises at least one promoter region operably connected to a nucleotide sequence encoding for a CRO repressor protein. In some embodiments, the sequence encoding for a CRO repressor protein is operably connected to a nuclear localization signal. In some embodiments, a polypeptide is provided that is encoded by a polynucleotide as provided for herein.

### CROSS-REFERENCE TO RELATED APPLICATIONS

This application claims priority to U.S. Provisional Application No. 63/292,956, filed on Dec. 22, 2021, which is incorporated by reference in its entirety.

### SEQUENCE LISTING

This application contains a Sequence Listing filed electronically as an XML file named “260034.000502 Sequence Listing XML”, which was created on Dec. 20, 2022 and is 28,884 bytes in size. The Sequence Listing is hereby incorporated by reference in its entirety.

### FIELD

Embodiments provided herein relate to transcriptional repressors and methods of using the same.

### BACKGROUND

Repressors are DNA or RNA binding proteins that bind to operator or silencer sequences to inhibit the expression of one or more genes. In molecular biology, repressor systems can be used to control the expression of a gene or genes of interest under experimental conditions. The two most commonly used inducible expression systems for this use are the Tet-Off and Tet-On systems. In the Tet-Off system, the tetracycline transactivator (tTA) fusion protein binds to DNA at specific TetO operator sequences to promote the expression of a gene of interest. When tetracyclines, such as doxycycline, are introduced to the experimental system, the tetracyclines bind tTA and prevent it from binding the TetO operator sequences, thereby shutting off transcription of the gene of interest. Conversely, in the Tet-On system, a mutant of the Tet repressor protein (TetR) dubbed reverse TetR (rTetR) is utilized to construct a reverse-tTA fusion protein (rtTA). rtTA binds specific TetO operator sequences only in the presence of tetracyclines, thus when a tetracycline is present it will bind rtTA, which then binds TetO and induces expression of the gene of interest.

An obvious drawback of the Tet-Off and Tet-On systems is that control of gene expression requires the presence of tetracyclines in one form or another. This is a major hurdle for development of in vivo inducible expression systems, as bioavailability of the tetracycline will be dependent on the tissue of interest. The requisite presence of tetracyclines can also be disadvantageous in vitro, where tetracyclines can disrupt protein translation in mitochondria. Thus, there is a need for improved transcriptional repressors and repressor systems that will not have the drawbacks of the above mentioned systems. The embodiments provided herein fulfill these needs as well as others.

### SUMMARY

In some embodiments, a polynucleotide is provided. In some embodiments, the polynucleotide comprises at least one promoter region operably connected to a nucleotide sequence encoding for a CRO repressor protein. In some embodiments, the sequence encoding for a CRO repressor protein is operably connected to a nuclear localization signal (“CRO-NLS protein”). In some embodiments, a polypeptide is provided that is encoded by a polynucleotide as provided for herein.

In some embodiments, a polypeptide, including an isolated polypeptide, is provided. In some embodiments, the polypeptide comprises a CRO repressor protein operably connected to a nuclear localization signal (“CRO-NLS protein”).

In some embodiments, a cell is provided, wherein the cell comprises a polynucleotide as provided for herein. In some embodiments, the cell further comprises a cargo polynucleotide. In some embodiments, the cargo polynucleotide comprises a nucleic acid sequence encoding for a molecule of interest. In some embodiments, the cargo polynucleotide further comprises a promoter region operably connected to the molecule of interest. In some embodiments, the cargo polynucleotide further comprises at least a first CRO repressor binding site. In some embodiments, the cargo polynucleotide comprises a nucleic acid sequence encoding for a molecule of interest, a promoter region operably connected to the molecule of interest, and at least a first CRO repressor binding site.

In some embodiments, a cell is provided, wherein the cell comprises a polypeptide as provided for herein. In some embodiments, the cell further comprises a cargo polynucleotide. In some embodiments, the cargo polynucleotide comprises a nucleic acid sequence encoding for a molecule of interest. In some embodiments, the cargo polynucleotide further comprises a promoter region operably connected to the molecule of interest. In some embodiments, the cargo polynucleotide further comprises at least a first CRO repressor binding site. In some embodiments, the cargo polynucleotide comprises a nucleic acid sequence encoding for a molecule of interest, a promoter region operably connected to the molecule of interest, and at least a first CRO repressor binding site.

In some embodiments, a virus is provided. In some embodiments, the virus comprises a cargo polynucleotide. In some embodiments, the cargo polynucleotide comprises a nucleic acid sequence encoding for a molecule of interest. In some embodiments, the cargo polynucleotide further comprises a promoter region operably connected to the molecule of interest. In some embodiments, the cargo polynucleotide further comprises at least a first CRO repressor binding site. In some embodiments, the cargo polynucleotide comprises a nucleic acid sequence encoding for a molecule of interest, a promoter region operably connected to the molecule of interest, and at least a first CRO repressor binding site.

In some embodiments, a method of making a virus comprising the cargo polynucleotide is provided. In some embodiments, the method comprises culturing a cell as provided for herein under conditions to produce the virus.

In some embodiments, a virus is provided. In some embodiments, the virus is prepared according to a method as provided for herein.

In some embodiments a plasmid is provided. In some embodiments, the plasmid comprises a polynucleotide as provided for herein.

In some embodiments, a cell is provided. In some embodiments, the cell comprises a polynucleotide as provided for herein. In some embodiments, the cell comprises a plasmid as provided for herein.

In some embodiments, a method of controlling expression of a molecule of interest is provided. In some embodiments, the method comprises contacting a host cell comprising a polynucleotide as provided for herein with a cargo polynucleotide comprising a nucleic acid sequence encoding for a molecule of interest, a promoter region operably connected to the molecule of interest, and at least a first CRO repressor binding site, wherein the expression of the molecule of interest is controlled by the binding of the polypeptide encoded for the polynucleotide as provided for herein to the CRO repressor binding site. In some embodiments, the method comprises contacting a host cell comprising a polypeptide as provided for herein with a cargo polynucleotide comprising a nucleic acid sequence encoding for a molecule of interest, a promoter region operably connected to the molecule of interest, and at least a first CRO repressor binding site, wherein the expression of the molecule of interest is controlled by the binding of the polypeptide as provided for herein to the CRO repressor binding site.

In some embodiments, a method of delivering a molecule of interest to a subject is provided. In some embodiments, the method comprises administering a virus as provided for herein to the subject.

In some embodiments, a method of inducing an immune response in a subject against a molecule of interest is provided. In some embodiments, the method comprises administering a virus as provided for herein to the subject, wherein the molecule of interest is a viral protein or a tumor antigen.

In some embodiments, a method of treating a disease is provided. In some embodiments, the method comprises administering a virus as provided for herein to a subject to treat the disease.

In some embodiments, a polypeptide is provided. In some embodiments, the polypeptide comprises a nuclear localization signal (NLS) linked or fused to a heterologous molecule. In some embodiments, the NLS is a polypeptide having a formula of NLS₁-Xₙ-NLS₂, wherein NLS₁ and NLS₂ can comprise the same or different NLS sequences, and Xₙ is a peptide linker such as described herein.

In some embodiments, a polynucleotide molecule is provided encoding for a polypeptide comprising a NLS as provided for herein.

In some embodiments, a plasmid is provided comprising the polynucleotide molecule encoding for a polypeptide comprising a NLS as provided for herein. In some embodiments, a cell is provided comprising the polynucleotide molecule encoding for a polypeptide comprising a NLS as provided for herein. In some embodiments, a virus is provided comprising the polynucleotide molecule encoding for a polypeptide comprising a NLS as provided for herein.

In some embodiments, a cell is provided comprising a polypeptide comprising a NLS as provided for herein.

In some embodiments, a method of transporting a heterologous molecule of interest to the nucleus of a cell is provided. In some embodiments, the method comprises contacting the cell with a polypeptide comprising a NLS as provided for herein. In some embodiments, the method comprises contacting the cell with a polynucleotide encoding for a polypeptide comprising a NLS as provided for herein under conditions sufficient to express the molecule in the cell. In some embodiments, the method comprises contacting the cell with a plasmid comprising a polynucleotide encoding for a polypeptide comprising a NLS as provided for herein under conditions sufficient to express the molecule in the cell. In some embodiments, the method comprises contacting the cell with a vector comprising a polynucleotide encoding for a polypeptide comprising a NLS as provided for herein.

### BRIEF DESCRIPTION OF THE FIGURES

FIG. 1 depicts a non-limiting examples of a CRO repressor protein as provided for herein.

### DETAILED DESCRIPTION

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20% or +10%, +5%, +1%, or ±0.1% from the specified value, as such variations are appropriate to perform the disclosed methods.

As used herein, the term “cargo” is meant to refer to any product that may be encoded by a nucleic acid molecule. As non-limiting examples, “cargo” may refer to an siRNA, an shRNA, a peptide, a polypeptide, a protein, a viral payload, a viral genome, or a combination thereof. In some embodiments, the cargo is a nucleic acid molecule encoding for any of the non-limiting examples.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of” or “consists.”

As used herein, the term “contacting” means bringing together of two elements in an in vitro system or an in vivo system. For example, “contacting” a vector with a cell or with an individual or patient or cell includes the administration of the vector to an individual or patient, such as a human, as well as, for example, introducing a compound into a sample containing a cellular or purified preparation containing the cell. As used herein, “contacting” can be synonymous with “delivering”. As used herein, “contacting” or “delivering” encompasses all necessary steps or methods. For example, “contacting” a vector with a cell would comprise nucleoporation, transfection, viral delivery, and the like.

“CRO repressor” as used herein refers to the Cro repressor protein of a bacteriophage. In bacteriophage, the Cro repressor acts to turn off early gene transcription during the lytic cycle. As used herein CRO repressor is not limited to a specific species of bacteriophage. Additionally, as used herein, “CRO repressor” is not limited to a specific CRO isoform, but encompasses all members of the Cro repressor family that perform a similar function. In some embodiments, the CRO repressor is the lambda bacteriophage CRO repressor protein.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune cell activation compared to the immune cell activation detected in the absence of the composition. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another. In some embodiments, the various domains or proteins provided for herein are linked or fused directly to one another or a linker sequences, such as a glycine/serine sequence link the two domains together.

“Heterologous” as used herein refers to a non-native nucleic acid or amino acid sequence that is introduced into a cell, organism, or system. The nucleic acid sequence can comprise a polynucleotide of any length. The amino acid sequence can comprise a peptide or polypeptide of any length.

“Identity” as used herein refers to the subunit sequence identity between two polymeric molecules such as between two nucleic acid or amino acid molecules, such as, between two polynucleotide or polypeptide molecules. When two amino acid sequences have the same residues at the same positions; e.g., if a position in each of two polypeptide molecules is occupied by an Arginine, then they are identical at that position. The identity or extent to which two amino acid or two nucleic acid sequences have the same residues at the same positions in an alignment is often expressed as a percentage. The identity between two amino acid or two nucleic acid sequences is a direct function of the number of matching or identical positions; e.g., if half of the positions in two sequences are identical, the two sequences are 50% identical; if 90% of the positions (e.g., 9 of 10), are matched or identical, the two amino acids sequences are 90% identical.

As used herein “located” is meant to give positional clarity in an amino acid or nucleic acid sequence. For example, a sequence X that is said to be located between a first portion A and a second portion B would yield the potential formulas A-X-B or B-X-A.

In a similar manner, the term “upstream” is meant to give further positional clarity in a nucleic acid or polynucleotide sequence. In the context of a polynucleotide sequence, a sequence X that is said to be located upstream of a first portion A would indicate that the sequence X is located prior to portion A such that the formula would read 5′-X-A-3′.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity can be measured/determined using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e3 and e100 indicating a closely related sequence. In some embodiments, sequence identity is determined by using BLAST with the default settings.

To the extent embodiments provided for herein, includes composition comprising various proteins, these proteins may, in some instances, comprise amino acid sequences that have sequence identity to the amino acid sequences disclosed herein. Therefore, in certain embodiments, depending on the particular sequence, the degree of sequence identity is preferably greater than 50% (e.g. 60%, 70%, 75%, 80%, 85%, 90%, 91%, 92%, 93%, 94%, 95%, 96%, 97%, 98%, 99% or more) to the SEQ ID NOs disclosed herein. These proteins may include homologs, orthologues, allelic variants and functional mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty-12 and gap extension penalty=1.

These proteins may, compared to the disclosed proteins, include one or more (e.g. 1, 2, 3,4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, Substitution of single amino acids within these families does not have a major effect on the biological activity. The proteins may have one or more (e.g. 1, 2, 3, 4, 5, 6, 7, 8, 9, 10, etc.) single amino acid deletions relative to the disclosed protein sequences. The proteins may also include one or more (e.g. 1, 2, 3, 4, 5, 6, 7, 8, 9, 10, etc.) insertions (e.g. each of 1, 2, 3, 4 or 5 amino acids) relative to the disclosed protein sequences.

As used herein, the phrase “in vivo” in reference to a cell being transduced, transfected or transformed in vivo, refers to a cell being transduced, transfected or transformed in the subject without the cells being removed from the subject before such cells are transduced, transfected or transformed.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell as provided herein. Molecules may be modified in many ways, including chemically, structurally, and functionally, such as mutations, substitutions, insertions, or deletions (e.g. internal deletions truncations). Cells may be modified through the introduction of nucleic acids or the expression of heterologous proteins.

By the term “modulating,” as used herein, is meant mediating an increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as, a human.

As used herein, the following abbreviations for the commonly occurring nucleic acid bases are used: “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “oligonucleotide” typically refers to short polynucleotides. It will be understood that when a nucleotide sequence is represented by a DNA sequence (i.e., A, T, C, G), this also provides the corresponding RNA sequence (i.e., A, U, C, G) in which “U” replaces “T.”

As used herein, the phrase “operably connected” or “operably linked” is defined as a first sequence having an impact on the translation, location, function, etc. of a second sequence. With respect to a promoter sequence and a sequence encoding a molecule of interest, the promoter sequence is considered to be operably linked if binding of the corresponding promoter induces transcription or translation of the molecule of interest. With respect to a nuclear localization signal and a molecule of interest, the nuclear localization signal is considered to be operably linked if presence of the nuclear localization signal induces translocation of the molecule of interest to the nucleus of a cell. The phrase “operably connected” or “operably linked” is not meant to be limiting in the proximity of the effector sequence (promoter, NLS, etc.) to the molecule of interest. The two sequences (effector and molecule of interest) can be directly fused together, or joined with a linker sequence of any appropriate length such that the effector sequence still has the desired effect on the molecule of interest.

The term “polynucleotide” as used herein is defined as a chain of nucleotides. Furthermore, nucleic acids are polymers of nucleotides. Thus, the terms “nucleic acids” and “polynucleotides” as used herein are interchangeable. As used herein polynucleotides include, but are not limited to, all nucleic acid sequences which are obtained by any methods available in the art, including, without limitation, recombinant methods, i.e., the cloning of nucleic acid sequences from a recombinant library or a cell genome, using cloning technology and PCR, and the like, and by synthetic means.

As used herein, the terms “peptide,” “polypeptide,” and “protein” are used interchangeably, and refer to a compound comprised of a plurality of amino acid residues covalently linked by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “self-cleaving peptide site” refers to a class of peptides which can induce ribosomal skipping during translation of a protein in a cell. These sites are found in a wide range of viral families and have been utilized in molecular biology to allow for at least two proteins of interest to be translated based on a single promoter with very high efficiency, wherein the first protein is not translated with a significantly higher efficiency than the second protein. This is in contrast to some traditional IRES sequences known in the art, where the second protein may be translated with far less efficiency than the first protein. Some specific self-cleaving peptide sites include the 2A peptides, which are 18-22 amino acid long peptide sequences. Examples of 2A peptides include T2A (derived from thosea asigna virus), P2A (derived from porcine teshovirus), E2A (derived from equine rhinitis A virus), and F2A (derived from foot and mouth disease virus).

The term “subject” includes living organisms, including those in which an immune response can be elicited (e.g., mammals). As used herein, the term “subject” or “patient” or “individual” may be used interchangeably. A “subject”, as used herein, may be a human or non-human mammal. Non-human mammals include, for example, livestock and pets, such as ovine, bovine, porcine, canine, non-human primates, feline and murine mammals. In some embodiments, the subject is human.

As used herein, the phrase “in need thereof” means that the subject (animal or mammal) has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into a cell. A “transfected” or “transformed” or “transduced” cell is one which has been transfected, transformed or transduced with exogenous nucleic acid. The cell includes the primary subject cell and its progeny. In some embodiments, the transfection, transformation, or transduction is performed or occurs in vivo.

To “treat” a disease as the term is used herein, means to reduce the frequency or severity of at least one sign or symptom of a disease or disorder experienced by a subject.

As used herein, the term “variant” when used in conjunction to an amino acid sequence refers to a sequence that is at least, or about, 85%, 90%, 91%, 92%, 93%, 94%, 95%, 96%, 97%, 98%, or 99% identical to the reference sequence. In some embodiments, the variant comprises 1, 2, 3, 4, 5, 6, 7, 8, 9, or 10 substitutions. In some embodiments, the substitution is a conservative substitution.

A “vector” is a composition of matter which comprises an isolated nucleic acid encoding a protein or a peptide. Numerous vectors are known in the art including, but not limited to, linear polynucleotides, plasmids, DNA, and RNA. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the embodiments can be presented in a range format. It should be understood that the description in range format is merely for convenience and brevity and should not be construed as an inflexible limitation. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Unless otherwise explicitly stated to the contrary, a range that is disclosed also includes the endpoints of the range.

Without being bound to any particular theory, the embodiments provided for herein have been found to control the production of viral particles such that alterations in host cell behavior or function including, but not limited to productivity, scalability or viability, are reduced and a higher titer of virus is achieved. Previously, production of viral particles in packaging cell lines was limited by the health of said cell line. By inserting transcription repressor binding sites within a growth-inhibitory or toxic viral payload and incorporating the corresponding transcription repressor into the genome of the packaging cell lines, production of recombinant virus was achievable. This allowed for decreased cytotoxicity to the packaging cell lines and an increased viral titer to be obtained.

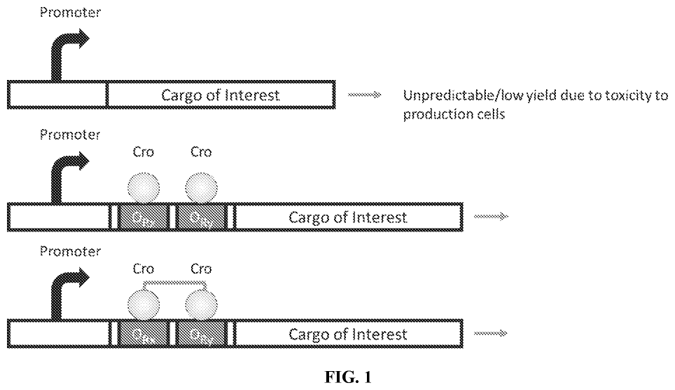

For example, as illustrated in FIG. 1, a cargo of interest, which can be referred to a heterologous molecule of interest or polynucleotide of interest, is under the control of a promoter. Because the promoter is not under any other control, this can lead to leakiness when producing, for example, a vector or virus, wherein the cargo of interest is expressed to the detriment of the production cell line. This can lead to unpredictable yield or low yield due to toxicity to production cells. The leakiness can be controlled by the CRO repressor protein, as provided for herein, which is illustrated in FIG. 1. This can be done, for example, with a monomeric CRO repressor protein or, for example, a dimeric CRO repressor protein. As illustrated in FIG. 1, the CRO repressor protein can bind to the CRO repressor binding site when the nucleic acid sequence is in the cell expressing the CRO repressor protein, thereby inhibiting the expression of the cargo of interest. When the nucleic acid molecule is expressed or is present in a cell without the CRO repressor protein, the cargo of interest can be expressed as regulated by the promoter. The promoter can be tissue or cell specific, or species specific.

Also provided herein are proteins or molecules comprising a nuclear localization signal (“NLS”). A NLS can be used to transport a molecule to the nucleus. In some embodiments, the NLS comprises a sequence of SEQ ID NO: 5 or as otherwise provided of herein.

### Compositions

Accordingly, described herein are polynucleotide molecules comprising a nucleotide sequence encoding for a CRO repressor protein. The CRO repressor protein utilized in the following embodiments is not limited to a specific isoform of CRO, but rather encompasses CRO repressor proteins of various bacteriophage species. In some embodiments, the CRO repressor protein is the lambda CRO repressor protein, such as described herein. In some embodiments, the polynucleotide molecules further comprise at least one promoter region operably connected to the nucleotide sequence encoding for a CRO repressor protein. In some embodiments, the at least one promoter region is located upstream of the sequence encoding the CRO repressor protein. In some embodiments, the polynucleotide molecules further comprise a nuclear localization signal (NLS) operably connected to the nucleotide sequence encoding for a CRO repressor protein. In some embodiments, the polynucleotide molecules comprise at least one promoter region operably connected to a nucleotide sequence encoding for a CRO repressor protein operably connected to a nuclear localization signal (hereafter referred to as “CRO-NLS” or “CRO-NLS protein”). In some embodiments, the at least one promoter region is located upstream of the sequence encoding the CRO-NLS protein. In the context of the present application, “operably connected” in regards to CRO and NLS can mean a CRO-NLS fusion protein, wherein the fusion protein may be a contiguous sequence with no spacer or linker region separating the CRO and the NLS. In some embodiments the fusion protein contains additional spacer or linker sequences separating the CRO and the NLS. The location of the NLS is not limited to the N terminus or the C terminus of the encoded protein. One skilled in the art will recognize that an NLS can also be added within the sequence encoding the protein of interest and not result in a deleterious effect on the native protein function. Thus, the present application is not limited to the NLS locations presented in embodiments herein, but also encompasses any NLS location that will i) result in CRO localization to the nucleus and ii) not hinder the native function of the CRO protein.

In some embodiments, the nucleotide sequence encoding for the CRO-NLS protein encodes a CRO repressor protein linked to the NLS by a linker. Accordingly, in some embodiments, the nucleotide sequence encodes a linker that is a peptide linker, such as a glycine/serine (GS) linker. In some embodiments, the GS linker comprises an amino acid sequence including but not limited to (GGGGS)ₙ(SEQ ID NO: 22), (GGGSS)ₙ(SEQ ID NO: 23), (GSGSG)ₙ(SEQ ID NO: 24), or (GSSG)ₙ(SEQ ID NO: 25), wherein each n is, independently, from 1 and 5, or a combination thereof. In some embodiments, n is 1. In some embodiments, n is 2. In some embodiments, n is 3. In some embodiments, n is 4. In some embodiments, n is 5. In some embodiments the linker is a cleavable linker. In some embodiments, the cleavable linker comprises a sequence of GPGSKLKSGFGGPGSRFRSGPG (SEQ ID NO: 1). In some embodiments, the linker is a non-cleavable linker. In some embodiments the non-cleavable linker comprises a sequence of AAGGTGGGSGGGTGGS (SEQ ID NO: 2). In some embodiments, the linker encoded by the polynucleotide is a peptide linker including, but not limited to (GGGGS)ₙ(SEQ ID NO: 22), (GGGSS)ₙ(SEQ ID NO: 23), (GSGSG)ₙ(SEQ ID NO: 24), (GSSG)ₙ(SEQ ID NO: 25), GPGSKLKSGFGGPGSRFRSGPG (SEQ ID NO: 1), or AAGGTGGGSGGGTGGS (SEQ ID NO: 2), or any combination thereof, wherein each n is, independently, 1-5.

In some embodiments, the linker encoded by the polynucleotide is a peptide linker including, but not limited to MXGXG, IXGXG, LXGXG, MXGGX, IXGGX, LXGGX, or any combination thereof, or any repetition thereof, wherein each X can be, independently, any amino acid. In some embodiments, the linker encoded by the polynucleotide is a peptide linker including, but not limited to, GPGPG (SEQ ID NO: 32), or any repetition thereof. In some embodiments, the linker encoded by the polynucleotide is a peptide linker including, but not limited to, GPGPG (SEQ ID NO: 32).

The NLS can be any NLS known to target a protein to the nucleus. In some embodiments, the NLS is a peptide NLS. In some embodiments, the NLS is a c-myc nuclear localization sequence with an amino acid sequence comprising PAAKRVKLD (SEQ ID NO: 3). In some embodiments, the NLS is the SV40 nuclear localization sequence with an amino acid sequence comprising PKKKRKV (SEQ ID NO: 4). In some embodiments, the NLS is a synthetic bipartite nuclear localization sequence. In some embodiments, the synthetic bipartite nuclear localization sequence is a combination of the c-myc nuclear localization sequence and the SV40 nuclear localization sequence. In some embodiments, the synthetic bipartite nuclear localization sequence has an amino acid sequence comprising PAAKRVKLDATESQDTGPPKKKRKV (SEQ ID NO: 5). In some embodiments, the NLS comprises an amino acid sequence of SEQ ID NO: 3, SEQ ID NO: 4, SEQ ID NO: 5, or any combination thereof. In some embodiments, the NLS comprises the synthetic bipartite NLS sequence and comprises an amino acid sequence of SEQ ID NO: 5.

In some embodiments, the CRO-NLS protein encoded by the nucleotide sequence comprises a monomer of a CRO repressor protein. In some embodiments, the CRO-NLS protein encoded by the nucleotide sequence comprises a dimer of a CRO repressor protein.

In the context of the present application, a “dimer” of a CRO repressor protein indicates that the CRO-NLS protein comprises two subunits of a CRO repressor protein. Each subunit can, independently, comprise an entire CRO repressor protein or an active fragment thereof. The two subunits can comprise the same or different CRO repressor proteins or active fragments thereof. In some embodiments, the two CRO subunits are linked together via a linker sequence. In some embodiments, the linker sequence is a peptide linker sequence. In some embodiments, the linker peptide sequence comprises an amino acid sequence as defined herein.

In the following embodiments, unless otherwise defined, “a CRO repressor protein” can comprise at least a monomer, a dimer, or an n-mer, wherein n is any number greater than 1 (e.g., 1, 2, 3, 4, or 5, or more), “a peptide linker” or “flexible linker moiety” can comprise a flexible peptide linker such as a GS linker as defined herein, or another linker as defined herein, or a cleavable linker as defined herein, or a non-cleavable linker as defined herein, and “at least one nuclear localization signal” can comprise a peptide NLS as defined herein. In some embodiments, the cleavable linker has the amino acid sequence SEQ ID NO: 1. In some embodiments, the non-cleavable linker has the amino acid sequence SEQ ID NO: 2.

In some embodiments, the CRO-NLS protein encoded by the polynucleotide comprises a polypeptide having a formula of X₂-L₂-X₃, wherein:
X₂ is a CRO repressor protein,
L₂ is a peptide linker, and
X₃ comprises at least one nuclear localization signal.

In further embodiments, the order of X₂, and X₃ can be rearranged such that the following formulas are available: X₂-L₂-X₃; or X₃-L₂-X₂, or any combination thereof.

In some embodiments, the CRO-NLS protein encoded by the polynucleotide comprises a polypeptide having a formula of X₁-L₁-X₂-L₂-X₃, wherein:
X₁ is an affinity binding domain,
X₂ is a CRO repressor protein,
X₃ comprises at least one nuclear localization signal or a synthetic bipartite nuclear localization signal, and
L₁ and L₂, are both, independently, flexible linker moieties, wherein L₁ and L₂ can be the same or different.

The order of X₁, L₁, X₂, L₂, and X₃ are not limited to the present embodiment described. In further embodiments, the position of any X provided can be substituted with any other X provided and the position of any L provided can be substituted with any other L provided. Thus a non-limiting example of an alternate formula would include X₁-L₂-X₃-L₁-X₂ and so forth. In further embodiments, the order of X₁, L₁, X₂, L₂ and X₃ can be rearranged such that any combination that forms a functional CRO repressor protein that is appropriately targeted to the nucleus is provided.

The affinity binding domain can be any affinity binding domain or epitope known in the art that will allow for any downstream processing, experimentation, or analysis with the polynucleotide. In some embodiments, the affinity binding domain comprises a peptide sequence encoded by the polynucleotide. In some embodiments, the affinity binding domain peptide sequence comprises a FLAG tag. In some embodiments, the affinity binding domain peptide sequence comprises a 3×FLAG tag. Other affinity binding domains are known in the art and can also be used. Non-limiting examples of affinity binding domains include CBP, HA, HBH, Myc, poly His, S-tag, TAP, V5, or combinations or active fragments thereof. In the following embodiments, unless otherwise defined, “an affinity binding domain” can comprise a peptide affinity binding domain as defined herein.

In some embodiments, the CRO-NLS protein encoded by the polynucleotide comprises a polypeptide having a formula of X₂-L₂-X₃-L₃-X₄, wherein:
X₂ comprises the CRO protein,
X₃ comprises at least one nuclear localization signal or a synthetic bipartite nuclear localization signal,
X₄ comprises the C-terminus of bacteriophage repressor lambda, and
L₂ and L₃ are each, independently, flexible linker moieties, wherein L₂ and L₃ are the same or different.

The order of X₂, L₂, X₃, L₃ and X₄ are not limited to the present embodiment described. In further embodiments, the position of any X provided can be substituted with any other X provided and the position of any L provided can be substituted with any other L provided. Thus a non-limiting example of an alternate formula would include X₃-L₃-X₂-L₂-X₄ and so forth. In further embodiments, the order of X₂, L₂, X₃, L₃ and X₄ can be rearranged such that any combination that forms a functional CRO repressor protein that is appropriately targeted to the nucleus is provided.

In some embodiments, the CRO-NLS protein encoded by the polynucleotide comprises a polypeptide having a formula of X₁-L₁-X₂-L₂-X₃-L₃-X₄, wherein, X₁ comprises an affinity binding domain,
X₂ comprises the CRO protein,
X₃ comprises at least one nuclear localization signal or a synthetic bipartite nuclear localization signal,
X₄ comprises the C-terminus of bacteriophage repressor lambda, and
L₁, L₂ and L₃ are each, independently, flexible linker moieties, wherein L₂ and L₃ are the same or different.

The order of X₁, L₁, X₂, L₂, X₃, L₃ and X₄ are not limited to the present embodiment described. In further embodiments, the position of any X provided can be substituted with any other X provided and the position of any L provided can be substituted with any other L provided. Thus a non-limiting example of an alternate formula would include X₁-L₂-X₃-L₁-X₂-L₃-X₄ and so forth. In further embodiments, the order of X₁, L₁, X₂, L₂, X₃, L₃ and X₄ can be rearranged such that any combination that forms a functional CRO repressor protein that is appropriately targeted to the nucleus is provided.

In some embodiments, the CRO-NLS protein encoded by the polynucleotide comprises a polypeptide having a formula of X₂-L₂-X₅-L₃-X₃, wherein:
X₂ comprises a first CRO protein,
X₅ comprises a second CRO protein,
X₃ comprises at least one nuclear localization signal, and
L₂ and L₃ are each, independently, flexible linker moieties, wherein L₂ and L₃ can be the same or different, and wherein L₂ may optionally be a non-cleavable linker.

The order of X₂, L₂, X₅, L₃ and X₃ are not limited to the present embodiment described. In further embodiments, the position of any X provided can be substituted with any other X provided and the position of any L provided can be substituted with any other L provided, as long as the components X₂-L₂-X₅ are in that order. Thus a non-limiting example of an alternate formula would include X₃-L₃-X₂-L₂-X₅ and so forth. In further embodiments, the order of X₂, L₂, X₅, L₃ and X₃ can be rearranged such that any combination that forms a functional CRO repressor protein that is appropriately targeted to the nucleus is provided.

In further embodiments, the at least one nuclear localization signal comprises more than one nuclear localization signal. In some embodiments, the more than one nuclear localization signal can be incorporated into the polypeptide formula at multiple locations. Thus, in some embodiments, a formula of X₂-L₃-X₃-L₂-X₅-L₃-X₃ might be obtained. However, in a similar manner as described herein, the specific order X₂, L₂, X₅, L₃ and X₃ is not limited to the embodiment provided, and the position of any X provided can be substituted with any other X provided and the position of any L provided can be substituted with any other L provided, as long as the components X₂-L₂-X₅ are in that order. Although the multiple nuclear localization signals here are represented by the same variable X₃, it should be understood that in embodiments where the more than one nuclear localization signal is incorporated into the polypeptide formula at multiple locations, each nuclear localization signal may, independently, comprise the same or different sequence identity to the other nuclear localization signals in the construct.

In some embodiments, the CRO-NLS protein encoded by the polynucleotide comprises a polypeptide having a formula of X₁-L₁-X₂-L₂-X₅-L₃-X₃, wherein:
X₁ comprises an affinity binding domain,
X₂ comprises a first CRO protein,
X₅ comprises a second CRO protein,
X₃ comprises at least one nuclear localization signal, and
L₁, L₂ and L₃ are each, independently, flexible linker moieties, wherein L₁, L₂ and L₃ can be the same or different, and wherein L₂ may optionally be a non-cleavable linker.

The order of X₁, L₁, X₂, L₂, X₅, L₃ and X₃ are not limited to the present embodiment described. In further embodiments, the position of any X provided can be substituted with any other X provided and the position of any L provided can be substituted with any other L provided, as long as the components X₂-L₂-X₅ are in that order. Thus a non-limiting example of an alternate formula would include X₁-L₃-X₃-L₁-X₂-L₂-X₅ and so forth. In further embodiments, the order of X₁, L₁, X₂, L₂, X₅, L₃ and X₃ can be rearranged such that any combination that forms a functional CRO repressor protein that is appropriately targeted to the nucleus is provided.

In further embodiments, the at least one nuclear localization signal comprises more than one nuclear localization signal. In some embodiments, the more than one nuclear localization signal can be incorporated into the polypeptide formula at multiple locations. Thus, in some embodiments, a formula of X₁-L₁-X₂-L₃-X₃-L₂-X₅-L₃-X₃ might be obtained. However, in a similar manner as described herein, the specific order X₁, L₁, X₂, L₂, X₅, L₃ and X₃ is not limited to the embodiment provided, and the position of any X provided can be substituted with any other X provided and the position of any L provided can be substituted with any other L provided, as long as the components X₂-L₂-X₅ are in that order. Although the multiple nuclear localization signals here are represented by the same variable X₃, it should be understood that in embodiments where the more than one nuclear localization signal is incorporated into the polypeptide formula at multiple locations, each nuclear localization signal may, independently, comprise the same or different sequence identity to the other nuclear localization signals in the construct.

In embodiments provided for herein, the polynucleotide encodes for a monomeric CRO protein. In some embodiments, the monomeric CRO protein comprises the amino acid sequence:

(SEQ ID NO: 6)
MEQRITLKDYAMRFGQTKTAKDLGVYQSAINKAIHAGRKIFLTIN
ADGSVYAEEVKPFPSNKKTTA

or an amino acid sequence substantially similar to SEQ ID NO: 6, or an active fragment thereof.

In embodiments provided for herein, the polynucleotide encodes for a CRO protein that further comprises the C-terminus of bacteriophage repressor lambda. In some embodiments, the C-terminus of bacteriophage repressor lambda comprises the amino acid sequence:

(SEQ ID NO: 7)
LRSEYEYPVFSHVQAGMFSPELRTFTKGDAERWVSTTKKASDSAF
WLEVEGNSMTAPTGSKPSFPDGMLILVDPEQAVEPGDFCIARLGG
DEFTFKKLIRDSGQVFLQPLNPQYPMIPCNESCSVVGKVIASQWP
EETFG.

or an amino acid sequence substantially similar to SEQ ID NO: 7, or an active fragment thereof.

In some embodiments, the CRO-NLS protein encoded for by the polynucleotide comprises an amino acid sequence having at least 85%, 86%, 87%, 88%, 89%, 90%, 91%, 92%, 93%, 94%, 95%, 96%, 97%, 98%, or 99% identity to SEQ ID NO: 8, or is identical to SEQ ID NO: 8

(SEQ ID NO: 8)
MDYKDHDGDYKDHDIDYKDDDDKGGGSSMEQRITLKDYAMRFGQT
KTAKDLGVYQSAINKAIHAGRKIFLTINADGSVYAEEVKPFPSNK
KTTAGSGSGPAAKRVKLD

or is an active fragment thereof.

In some embodiments, the nucleotide sequence encoding for the CRO-NLS protein comprises a nucleic acid sequence having at least 85%, 86%, 87%, 88%, 89%, 90%, 91%, 92%, 93%, 94%, 95%, 96%, 97%, 98%, or 99% identity to SEQ ID NO: 9, or comprises the sequence of SEQ ID NO: 9

(SEQ ID NO: 9)
ATGGATTACAAGGACCACGACGGTGACTATAAAGACCATGATATT
GATTACAAAGATCATGACGACAAGGGCGGGGGAAGTAGTATGGAA
CAGCGGATAACTTTGAAAGACTATGCAATGCGCTTCGGCCAAACC
AAGACTGCTAAAGACTTGGGTGTGTACCAAAGTGCAATAAATAAA
GCTATCCACGCCGGGAGGAAGATCTTTCTCACGATAAACGCTGAC
GGCTCCGTGTATGCCGAAGAAGTGAAACCTTTCCCTTCTAATAAA
AAGACCACTGCGGGTTCCGGCAGTGGCCCGGCTGCAAAGCGGGTC
AAACTTGATTAA

or is an active fragment thereof. The sequence of SEQ ID NO: 9 (as well as other nucleotide sequences provided for herein) is an exemplary sequence and is not meant to be limiting in any way. Due to the degenerate nature of codons, other nucleic acid molecules can be used. In some embodiments, the nucleic acid molecule is codon optimized for expression in a bacterial system.

In some embodiments, the nucleic acid molecule is codon optimized for expression in a eukaryotic system or cell. In some embodiments, the nucleic acid molecule is a DNA or RNA molecule that encodes a polypeptide as provided for herein. In some embodiments, the RNA molecule is a mRNA molecule.

In some embodiments, the CRO-NLS protein encoded for by the polynucleotide comprises an amino acid sequence having at least 85%, 86%, 87%, 88%, 89%, 90%, 91%, 92%, 93%, 94%, 95%, 96%, 97%, 98%, or 99% identity to SEQ ID NO: 10, or is identical to SEQ ID NO: 10

(SEQ ID NO: 10)
MDYKDHDGDYKDHDIDYKDDDDKGGGSSMEQRITLKDYAMRFGQT
KTAKDLGVYQSAINKAIHAGRKIFLTINADGSVYAEEVKPFPSNK
KTTAGSGSGPAAKRVKLDATESQDTGPPKKKRKVGSSGLRSEYEY
PVFSHVQAGMFSPELRTFTKGDAERWVSTTKKASDSAFWLEVEGN
SMTAPTGSKPSFPDGMLILVDPEQAVEPGDFCIARLGGDEFTFKK
LIRDSGQVFLQPLNPQYPMIPCNESCSVVGKVIASQWPEETFG

or is an active fragment thereof.

In some embodiments, the nucleotide sequence encoding for the CRO-NLS protein comprises a nucleic acid sequence having at least 85%, 86%, 87%, 88%, 89%, 90%, 91%, 92%, 93%, 94%, 95%, 96%, 97%, 98%, or 99% identity to SEQ ID NO: 11, or comprises the sequence of SEQ ID NO: 11

(SEQ ID NO: 11)
ATGGACTACAAGGACCATGATGGTGATTATAAAGACCACGATATA
GACTACAAAGACGACGATGATAAGGGCGGGGGAAGTAGTATGGAA
CAGCGCATAACGTTGAAAGACTACGCAATGCGATTCGGTCAAACA
AAGACGGCTAAAGACTTGGGTGTTTACCAGAGTGCGATAAACAAG
GCCATCCATGCGGGAAGAAAGATATTTTTGACGATAAATGCAGAT
GGCTCCGTTTACGCGGAGGAAGTCAAGCCCTTTCCCTCAAATAAA
AAAACAACGGCTGGTTCCGGCAGTGGCCCTGCCGCCAAACGGGTG
AAACTGGACGCGACAGAGTCACAGGACACCGGCCCACCAAAGAAA
AAAAGAAAGGTGGGCAGTTCCGGGCTCCGAAGCGAATACGAGTAC
CCGGTGTTCTCCCATGTACAGGCCGGAATGTTTTCTCCGGAACTC
AGAACCTTCACGAAAGGCGATGCGGAGAGGTGGGTGAGTACCACC
AAAAAGGCCTCTGACAGTGCTTTTTGGCTTGAAGTAGAAGGGAAC
TCCATGACTGCGCCGACCGGATCTAAACCGAGTTTCCCGGATGGA
ATGCTCATCCTGGTCGATCCTGAACAAGCTGTTGAACCGGGTGAT
TTCTGTATCGCCCGCCTGGGGGGTGACGAGTTCACCTTCAAAAAG
CTCATTCGGGACTCCGGACAAGTTTTTTTGCAACCGCTTAATCCC
CAGTACCCTATGATTCCCTGCAATGAGTCCTGCTCTGTAGTGGGT
AAAGTGATAGCTAGTCAGTGGCCGGAAGAAACATTCGGGTAA

or is an active fragment thereof. As detailed herein, due to the degenerate nature of codons, the sequence of SEQ ID NO: 11 represents a non-limiting exemplary sequence, and alternate nucleic acid molecules can be utilized to obtain the same product. These alternate nucleic acid molecules are contained within the scope of the present application.

In some embodiments, the CRO-NLS protein encoded for by the polynucleotide comprises an amino acid sequence having at least 85%, 86%, 87%, 88%, 89%, 90%, 91%, 92%, 93%, 94%, 95%, 96%, 97%, 98%, or 99% identity to SEQ ID NO: 12, or is identical to SEQ ID NO: 12

(SEQ ID NO: 12)
MDYKDHDGDYKDHDIDYKDDDDKGGGSSMEQRITLKDYAMRFGQT
KTAKDLGVYQSAINKAIHAGRKIFLTINADGSVYAEEVKPFPSNK
KTTAAAGGTGGGSGGGTGGSMEQRITLKDYAMRFGQTKTAKDLGV
YQSAINKAIHAGRKIFLTINADGSVYAEEVKPFPSNKKTTAGSGS
GPAAKRVKLD

or is an active fragment thereof.

In some embodiments, the nucleotide sequence encoding for the CRO-NLS protein comprises a nucleic acid sequence having at least 85%, 86%, 87%, 88%, 89%, 90%, 91%, 92%, 93%, 94%, 95%, 96%, 97%, 98%, or 99% identity to SEQ ID NO: 13, or comprises the sequence of SEQ ID NO: 13

(SEQ ID NO: 13)
ATGGACTACAAAGACCATGATGGGGACTATAAGGATCACGACATA
GACTATAAGGACGATGATGACAAGGGCGGAGGAAGCAGCATGGAA
CAGAGAATCACACTCAAAGACTATGCAATGAGATTCGGTCAGACG
AAAACTGCCAAGGATCTCGGCGTCTACCAAAGCGCGATCAACAAA
GCAATTCATGCGGGACGAAAAATTTTTCTGACGATAAATGCTGAT
GGATCAGTGTACGCAGAAGAGGTAAAGCCTTTCCCGTCAAACAAG
AAAACGACAGCGGCAGCGGGCGGCACGGGGGGAGGTTCCGGGGGC
GGGACAGGTGGCTCTATGGAGCAACGCATTACTCTGAAGGACTAC
GCCATGAGATTCGGGCAGACAAAGACTGCTAAGGATTTGGGGGTA
TATCAATCAGCAATCAATAAGGCAATACATGCAGGGCGCAAAATT
TTTCTTACGATAAACGCTGATGGAAGTGTATACGCTGAAGAAGTA
AAACCTTTCCCAAGTAATAAGAAAACGACCGCCGGTAGCGGGAGC
GGACCAGCCGCCAAGCGGGTAAAACTCGATTAA

or is an active fragment thereof. As detailed herein, due to the degenerate nature of codons, the sequence of SEQ ID NO: 13 represents a non-limiting exemplary sequence, and alternate nucleic acid molecules can be utilized to obtain the same product. These alternate nucleic acid molecules are contained within the scope of the present application.

In some embodiments, the CRO-NLS protein encoded for by the polynucleotide comprises an amino acid sequence having at least 85%, 86%, 87%, 88%, 89%, 90%, 91%, 92%, 93%, 94%, 95%, 96%, 97%, 98%, or 99% identity to SEQ ID NO: 14, or is identical to SEQ ID NO: 14

(SEQ ID NO: 14)
MDYKDHDGDYKDHDIDYKDDDDKGGGSSMEQRITLKDYAMRFGQT
KTAKDLGVYQSAINKAIHAGRKIFLTINADGSVYAEEVKPFPSNK
KTTAGPGSKLKSGFGGPGSRFRSGPGMEQRITLKDYAMRFGQTKT
AKDLGVYQSAINKAIHAGRKIFLTINADGSVYAEEVKPFPSNKKT
TAGSGSGPAAKRVKLD

or is an active fragment thereof.

In some embodiments, the nucleotide sequence encoding for the CRO-NLS protein comprises a nucleic acid sequence having at least 85%, 86%, 87%, 88%, 89%, 90%, 91%, 92%, 93%, 94%, 95%, 96%, 97%, 98%, or 99% identity to SEQ ID NO: 15, or comprises the sequence of SEQ ID NO: 15

(SEQ ID NO: 15)
ATGGACTACAAAGACCATGATGGGGACTATAAGGATCACGACATA
GACTATAAGGACGATGATGACAAGGGCGGAGGAAGCAGCATGGAA
CAGAGAATCACACTCAAAGACTATGCAATGAGATTCGGTCAGACG
AAAACTGCCAAGGATCTCGGCGTCTACCAAAGCGCGATCAACAAA
GCAATTCATGCGGGACGAAAAATTTTTCTGACGATAAATGCTGAT
GGATCAGTGTACGCAGAAGAGGTAAAGCCTTTCCCGTCAAACAAG
AAAACGACAGCGGGACCTGGAAGCAAACTTAAAAGCGGATTTGGA
GGGCCCGGGTCACGCTTCCGCTCTGGACCGGGCATGGAGCAACGC
ATTACTCTGAAGGACTACGCCATGAGATTCGGGCAGACAAAGACT
GCTAAGGATTTGGGGGTATATCAATCAGCAATCAATAAGGCAATA
CATGCAGGGCGCAAAATTTTTCTTACGATAAACGCTGATGGAAGT
GTATACGCTGAAGAAGTAAAACCTTTCCCAAGTAATAAGAAAACG
ACCGCCGGTAGCGGGAGCGGACCAGCCGCCAAGCGGGTAAAACTC
GATTAA

or is an active fragment thereof. As detailed herein, due to the degenerate nature of codons, the sequence of SEQ ID NO: 15 represents a non-limiting exemplary sequence, and alternate nucleic acid molecules can be utilized to obtain the same product. These alternate nucleic acid molecules are contained within the scope of the present application.

In some embodiments, a polypeptide is provided. In some embodiments, the polypeptide is encoded by the polynucleotide molecules as described herein. In some embodiments, the polypeptide comprises a CRO repressor protein operably connected to a nuclear localization signal (hereafter referred to as “CRO-NLS” or “CRO-NLS protein”). In some embodiments, the nuclear localization signal comprises any amino acid sequence known to target the polypeptide to the nucleus of a cell. In some embodiments, the NLS comprises an amino acid sequence of SEQ ID NO: 3, SEQ ID NO: 4, SEQ ID NO: 5, or any combination thereof. In some embodiments, the NLS comprises an amino acid sequence of SEQ ID. NO. 5. In some embodiments, the NLS is an NLS as provided for herein.

In some embodiments, the CRO repressor protein is linked to the NLS by a linker peptide. In some embodiments, the linker peptide comprises a glycine/serine linker, including, but not limited to, (GGGGS)ₙ(SEQ ID NO: 22), (GGGSS)ₙ(SEQ ID NO: 23), (GSGSG)ₙ(SEQ ID NO: 24), (GSSG)ₙ(SEQ ID NO: 25), GPGSKLKSGFGGPGSRFRSGPG (SEQ ID NO: 1), or AAGGTGGGSGGGTGGS (SEQ ID NO: 2), or any combination thereof, wherein each n is, independently, 1-5. In some embodiments, the linker peptide comprises an amino acid sequence of MXGXG, IXGXG, LXGXG, MXGGX, IXGGX, LXGGX, or any combination thereof, or any repetition thereof, wherein X can be, independently, any amino acid. In some embodiments, the linker peptide comprises an amino acid sequence of GPGPG (SEQ ID NO: 32), or any repetition thereof. In some embodiments, the linker peptide comprises an amino acid sequence of GPGPG (SEQ ID NO: 32). In some embodiments, the linker peptide is a linker peptide as provided for herein.

In some embodiments, the polypeptide CRO-NLS comprises a monomeric CRO repressor protein. In some embodiments, the polypeptide CRO-NLS comprises a dimeric CRO repressor protein.

In the following embodiments, unless otherwise defined, “a CRO repressor protein” can comprise a monomer or a dimer, “a peptide linker” or “flexible linker moiety” can comprise a flexible peptide linker such as a GS linker as defined herein, or another linker as defined herein, or a cleavable linker as defined herein, or a non-cleavable linker as defined herein, “at least one nuclear localization signal” can comprise a peptide NLS as defined herein, and “an affinity binding domain” can comprise a peptide affinity binding domain as defined herein. In some embodiments, the cleavable linker has the amino acid sequence SEQ ID NO: 1. In some embodiments, the non-cleavable linker has the amino acid sequence SEQ ID NO: 2.

In some embodiments, the CRO-NLS protein comprises a polypeptide having a formula of X₂-L₂-X₃, wherein:
X₂ is a CRO repressor protein,
L₂ is a peptide linker, and
X₃ comprises at least one nuclear localization signal.

In further embodiments, the order of X₂, and X₃ can be rearranged such that the following formulas are available: X₂-L₂-X₃; or X₃-L₂-X₂, or any combination thereof.

In some embodiments, the CRO-NLS protein comprises a polypeptide having a formula of X₁-L₁-X₂-L₂-X₃, wherein:
X₁ is an affinity binding domain,
X₂ is a CRO repressor protein,
X₃ comprises at least one nuclear localization signal or a synthetic bipartite nuclear localization signal, and
L₁ and L₂, are both, independently, flexible linker moieties, wherein L₁ and L₂ can be the same or different.

The order of X₁, L₁, X₂, L₂, and X₃ are not limited to the present embodiment described. In further embodiments, the position of any X provided can be substituted with any other X provided and the position of any L provided can be substituted with any other L provided. Thus a non-limiting example of an alternate formula would include X₁-L₂-X₃-L₁-X₂ and so forth. In further embodiments, the order of X₁, L₁, X₂, L₂ and X₃ can be rearranged such that any combination that forms a functional CRO repressor protein that is appropriately targeted to the nucleus is provided.

In some embodiments, the CRO-NLS protein comprises a polypeptide having a formula of X₂-L₂-X₃-L₃-X₄, wherein:
X₂ comprises the CRO protein,
X₃ comprises at least one nuclear localization signal or a synthetic bipartite nuclear localization signal,
X₄ comprises the C-terminus of bacteriophage repressor lambda, and
L₂ and L₃ are each, independently, flexible linker moieties, wherein L₂ and L₃ are the same or different.

The order of X₂, L₂, X₃, L₃ and X₄ are not limited to the present embodiment described. In further embodiments, the position of any X provided can be substituted with any other X provided and the position of any L provided can be substituted with any other L provided. Thus a non-limiting example of an alternate formula would include X₃-L₃-X₂-L₂-X₄ and so forth. In further embodiments, the order of X₂, L₂, X₃, L₃ and X₄ can be rearranged such that any combination that forms a functional CRO repressor protein that is appropriately targeted to the nucleus is provided.

In some embodiments, the CRO-NLS protein comprises a polypeptide having a formula of X₁-L₁-X₂-L₂-X₃-L₃-X₄, wherein,
X₁ comprises an affinity binding domain,
X₂ comprises the CRO protein,
X₃ comprises at least one nuclear localization signal or a synthetic bipartite nuclear localization signal,
X₄ comprises the C-terminus of bacteriophage repressor lambda, and
L₁, L₂ and L₃ are each, independently, flexible linker moieties, wherein L₂ and L₃ are the same or different.

The order of X₁, L₁, X₂, L₂, X₃, L₃ and X₄ are not limited to the present embodiment described. In further embodiments, the position of any X provided can be substituted with any other X provided and the position of any L provided can be substituted with any other L provided. Thus a non-limiting example of an alternate formula would include X₁-L₂-X₃-L₁-X₂-L₃-X₄ and so forth. In further embodiments, the order of X₁, L₁, X₂, L₂, X₃, L₃ and X₄ can be rearranged such that any combination that forms a functional CRO repressor protein that is appropriately targeted to the nucleus is provided.

In some embodiments, the CRO-NLS protein comprises a polypeptide having a formula of X₂-L₂-X₅-L₃-X₃, wherein:
X₂ comprises a first CRO protein,
X₅ comprises a second CRO protein,
X₃ comprises at least one nuclear localization, and
L₂ and L₃ are each, independently, flexible linker moieties, wherein L₂ and L₃ can be the same or different, and wherein L₂ may optionally be a non-cleavable linker.

The order of X₂, L₂, X₅, L₃ and X₃ are not limited to the present embodiment described. In further embodiments, the position of any X provided can be substituted with any other X provided and the position of any L provided can be substituted with any other L provided, as long as the components X₂-L₂-X₅ are in that order. Thus a non-limiting example of an alternate formula would include X₃-L₃-X₂-L₂-X₅ and so forth. In further embodiments, the order of X₂, L₂, X₅, L₃ and X₃ can be rearranged such that any combination that forms a functional CRO repressor protein that is appropriately targeted to the nucleus is provided.

In further embodiments, the at least one nuclear localization signal comprises more than one nuclear localization signal. In some embodiments, the more than one nuclear localization signal can be incorporated into the polypeptide formula at multiple locations. Thus, in some embodiments, a formula of X₂-L₃-X₃-L₂-X₅-L₃-X₃ might be obtained. However, in a similar manner as described herein, the specific order X₂, L₂, X₅, L₃ and X₃ is not limited to the embodiment provided, and the position of any X provided can be substituted with any other X provided and the position of any L provided can be substituted with any other L provided, as long as the components X₂-L₂-X₅ are in that order. Although the multiple nuclear localization signals here are represented by the same variable X₃, it should be understood that in embodiments where the more than one nuclear localization signal is incorporated into the polypeptide formula at multiple locations, each nuclear localization signal may, independently, comprise the same or different sequence identity to the other nuclear localization signals in the construct.

In some embodiments, the CRO-NLS protein comprises a polypeptide having a formula of X₁-L₁-X₂-L₂-X₅-L₃-X₃, wherein:
X₁ comprises an affinity binding domain, X₂ comprises a first CRO protein,
X₅ comprises a second CRO protein,
X₃ comprises at least one nuclear localization, and
L₁, L₂ and L₃ are each, independently, flexible linker moieties, wherein L₁, L₂ and L₃ can be the same or different, and wherein L₂ may optionally be a non-cleavable linker.

The order of X₁, L₁, X₂, L₂, X₅, L₃ and X₃ are not limited to the present embodiment described. In further embodiments, the position of any X provided can be substituted with any other X provided and the position of any L provided can be substituted with any other L provided, as long as the components X₂-L₂-X₅ are in that order. Thus a non-limiting example of an alternate formula would include X₁-L₃-X₃-L₁-X₂-L₂-X₅ and so forth. In further embodiments, the order of X₁, L₁, X₂, L₂, X₅, L₃ and X₃ can be rearranged such that any combination that forms a functional CRO repressor protein that is appropriately targeted to the nucleus is provided.

In further embodiments, the at least one nuclear localization signal comprises more than one nuclear localization signal. In some embodiments, the more than one nuclear localization signal can be incorporated into the polypeptide formula at multiple locations. Thus, in some embodiments, a formula of X₁-L₁-X₂-L₃-X₃-L₂-X₅-L₃-X₃ might be obtained. However, in a similar manner as described herein, the specific order X₁, L₁, X₂, L₂, X₅, L₃ and X₃ is not limited to the embodiment provided, and the position of any X provided can be substituted with any other X provided and the position of any L provided can be substituted with any other L provided, as long as the components X₂-L₂-X₅ are in that order. Although the multiple nuclear localization signals here are represented by the same variable X₃, it should be understood that in embodiments where the more than one nuclear localization signal is incorporated into the polypeptide formula at multiple locations, each nuclear localization signal may, independently, comprise the same or different sequence identity to the other nuclear localization signals in the construct.

In some embodiments, the first CRO protein as represented by X₂ in the above formula and the second CRO protein as represented by X₅ in the above formulas comprise the same amino acid sequence. In some embodiments, first CRO protein as represented by X₂ in the above formula and the second CRO protein as represented by X₅ in the above formulas comprise the same protein. In some embodiments, first CRO protein as represented by X₂ in the above formula and the second CRO protein as represented by X₅ in the above formulas comprise a different amino acid sequence. In some embodiments, the first CRO protein as represented by X₂ in the above formula and the second CRO protein as represented by X₅ in the above formulas comprise different CRO proteins.

In embodiments provided for herein, the CRO-NLS polypeptide comprises a monomeric CRO protein. In some embodiments, the monomeric CRO protein comprises the amino acid sequence of SEQ ID NO: 6, or an amino acid sequence substantially similar to SEQ ID NO: 6, or an active fragment thereof.

In some embodiments, the NLS sequence of the CRO-NLS polypeptide comprises an amino acid sequence of SEQ ID NO: 3, SEQ ID NO: 4, or SEQ ID NO: 5, or any combination thereof. In some embodiments, the NLS sequence of the CRO-NLS polypeptide comprises an amino acid sequence of SEQ ID NO: 5.

In some embodiments, the linker sequences as represented by L₁, L₂, and L₃ in the above formulas, as applicable, each, independently, comprise the amino acid sequences of SEQ ID NO: 22, SEQ ID NO: 23, SEQ ID NO: 24, SEQ ID NO: 25, SEQ ID NO: 1, or SEQ ID NO: 2, or any combination thereof, wherein each n is, independently, 1-5. In some embodiments, the linker sequences as represented by L₁, L₂, and L₃ in the above formulas, as applicable, each, independently, comprise the amino acid sequences of SEQ ID NO: 26, SEQ ID NO: 27, SEQ ID NO: 28, SEQ ID NO: 29, SEQ ID NO: 30, SEQ ID NO: 31, or any combination thereof, wherein X is any amino acid. In some embodiments, the linker sequences as represented by L₁, L₂, and L₃ in the above formulas, as applicable, each, independently, comprise the amino acid sequence of SEQ ID NO: 31.

In some embodiments, the linker sequence represented by L₂ in the above formulas comprise the amino acid sequence of SEQ ID NO: 1 or SEQ ID NO: 2.

In embodiments provided for herein, the CRO-NLS polypeptide further comprises the C-terminus of bacteriophage repressor lambda. In some embodiments, the C-terminus of bacteriophage repressor lambda comprises the amino acid sequence of SEQ ID NO: 7, or an amino acid sequence substantially similar to SEQ ID NO: 7, or an active fragment thereof.

In some embodiments, the CRO-NLS polypeptide further comprises an affinity binding domain. In some embodiments, the affinity binding domain is a peptide affinity binding domain as provided for herein.

In some embodiments, the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 8, or is identical to SEQ ID NO: 8, or is an active fragment thereof.

In some embodiments, the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 10, or is identical to SEQ ID NO: 10, or is an active fragment thereof. In some embodiments, the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 12, or is identical to SEQ ID NO: 12, or is an active fragment thereof.

In some embodiments, the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 14, or is identical to SEQ ID NO: 14, or is an active fragment thereof.

In some embodiments, a cell is provided. In some embodiments the cell comprises a polynucleotide as described herein. In some embodiments, the cell comprises a cargo polynucleotide comprising a nucleic acid sequence encoding for a molecule of interest. In some embodiments, the cargo polynucleotide further comprises a promoter region operably connected to the molecule of interest. In some embodiments, the cargo polynucleotide comprises a nucleic acid sequence encoding for a molecule of interest and at least a first CRO repressor binding site. In some embodiments, the cargo polynucleotide comprises a nucleic acid sequence encoding for a molecule of interest, a promoter region operably connected to the molecule of interest, and at least a first CRO repressor binding site. In some embodiments, the cell comprises a polynucleotide encoding a CRO-NLS protein as described herein and further comprises a cargo polynucleotide comprises a nucleic acid sequence encoding for a molecule of interest, a promoter region operably connected to the molecule of interest, and at least a first CRO repressor binding site.

In some embodiments, the promoter region of the cargo polynucleotide is located upstream of the sequence encoding the molecule of interest.

In some embodiments, the cell comprises a polynucleotide encoding a CRO-NLS protein as described herein and a cargo polynucleotide. In the context of the present application, “cargo polynucleotide” is considered to be synonymous to a “second polynucleotide” or an “additional polynucleotide” if the cargo nucleotide is in the presence of another heterologous polynucleotide.

In some embodiments, a cell is provided comprising a CRO-NLS polypeptide as described herein. In some embodiments, the cell further comprises a cargo polynucleotide comprising a nucleic acid sequence encoding for a molecule of interest. In some embodiments, the cargo polynucleotide further comprises a promoter region operably connected to the molecule of interest. In some embodiments, the cargo polynucleotide comprises a nucleic acid sequence encoding for a molecule of interest and at least a first CRO repressor binding site. In some embodiments, the cargo polynucleotide comprises a nucleic acid sequence encoding for a molecule of interest, a promoter region operably connected to the molecule of interest, and at least a first CRO repressor binding site.

In some embodiments, the CRO repressor binding site of the cargo polynucleotide is operably connected to the promoter region of the cargo polynucleotide, such that when in the presence of a CRO protein, the CRO protein binds to the CRO repressor binding site and inhibits the expression of the molecule of interest. In some embodiments, the CRO protein binding to the CRO repressor binding site inhibits the transcription of the molecule of interest.

In some embodiments, the CRO protein is the CRO-NLS protein as described herein. In some embodiments, the NLS signal of the CRO-NLS protein directs the CRO-NLS protein to the nucleus, where it can then bind the CRO repressor binding site of the cargo polynucleotide and inhibit the expression of the molecule of interest.

In some embodiments, the cargo polynucleotide comprises a 5′ adenoviral ITR and a 3′ adenoviral ITR, wherein the 5′ ITR and 3′ ITR flank the nucleic acid sequence encoding for the molecule of interest, the promoter region operably connected to the molecule of interest, and the least a first CRO repressor binding site.

In some embodiments, the at least a first CRO repressor binding site of the cargo polynucleotide is located upstream of the sequence encoding for the molecule of interest. In some embodiments, the at least a first CRO repressor binding site is located upstream of the promoter region of the cargo polynucleotide. Thus, in the presence of a CRO or CRO-NLS protein, CRO or CRO-NLS will substantially bind to the CRO repressor binding site and prevent the promoter from initiating transcription of the molecule of interest.

In some embodiments, the at least a first CRO repressor binding site is located between the promoter region and the sequence encoding for the molecule of interest of the cargo polynucleotide. Thus, in the presence of a CRO or CRO-NLS protein, CRO or CRO-NLS will substantially bind to the CRO repressor binding site and stop the promoter from initiating transcription of the molecule of interest.

In some embodiments, the at least a first CRO repressor binding site is located within the promoter region of the carbo polynucleotide. Thus, in the presence of a CRO or CRO-NLS protein, CRO or CRO-NLS will substantially bind to the CRO repressor binding site and prevent the promoter from binding to the promoter region, thus preventing the promoter from initiating transcription of the molecule of interest.

In embodiments as provided for herein, the at least a first CRO repressor binding site is selected from the group consisting of rightward operators of a bacteriophage and leftward operators of a bacteriophage. The repressor binding site is not limited in that it is derived from a specific bacteriophage. The CRO repressor binding site may be derived from, for example, any bacteriophage of the order Caudovirales, Timlovirales, Mindivirales, Petitvirales, Tubulavirales, and Norzivirales. These would include any bacteriophage derived from, for example, a family selected from Ackermannviridae, Autographiviridae, Chaseviridae, Demerecviridae, Drexlerviridae, Guenliviridae, Herelleviridae, Myoviridae, Siphoviridae, Podoviridae,
Rountreeviridae, Salasmaviridae, Schitoviridae, Zobellviridae, Blumeviridae, Steitzviridae, Cystoviridae, Inoviridae, Paulinoviridae, Plectroviridae, Atkinsviridae, Duinviridae, Fiersviridae, and Solspiviridae. Non limiting examples of bacteriophage are included in these orders and families include 186 phage, bacteriophage qCb5, G4 phage, M13 phage, MS2 phage, T4 phage, Mu phage, P1 phage, P2 phage, P4 phage, R17 phage, λphage, T2 phage, T5 phage, HK97 phage, N4 phage, N15 phage, T7 phage, T3 phage, T12 phage, @6 phage, @29 phage, @X 174, Qβ phage and P22 phage.

In some embodiments, the at least a first CRO repressor binding site is selected from the group consisting of rightward operators of bacteriophage λ and leftward operators of bacteriophage λ. In some embodiments, the at least a first CRO repressor binding site is selected from the group consisting of a bacteriophage A leftward operator 1 (O_{L1}), a bacteriophage λ leftward operator 2 (O_{L2}), a bacteriophage λ leftward operator 3 (O_{L3}), a bacteriophage λ rightward operator 1 (O_{R1}), a bacteriophage λ rightward operator 2 (O_{R2}), and a bacteriophage λ rightward operator 3 (O_{R3}).

Certain embodiments herein describe the use of chimeric repressor molecules and therefore certain modifications and combinations can be used to increase or decrease binding affinity of the repressor molecules to the operator sequences.

Accordingly, in some embodiments, the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 16, or is identical to SEQ ID NO: 16:

(SEQ ID NO: 16)
5′-TATCACCGCCAGTGGTAT-3′;

or is an active fragment thereof.

In some embodiments, the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 17, or is identical to SEQ ID NO: 17:

(SEQ ID NO: 17)
5′-TCAACACCGCCAGAGATAA-3′;

or is an active fragment thereof.

In some embodiments, the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 18, or is identical to SEQ ID NO: 18:

(SEQ ID NO: 18)
5′-TTATCACCGCAGATGGTTAT-3′;

or is an active fragment thereof.

In some embodiments, the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19, or is identical to SEQ ID NO: 19:

(SEQ ID NO: 19)
5′-TTATCACCGCCAGAGGTAA-3′;

or is an active fragment thereof.

In some embodiments, the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 20, or is identical to SEQ ID NO: 20:

(SEQ ID NO: 20)
5′-TCAACACGCACGGTGTTAG-3′;

or is an active fragment thereof.

In some embodiments, the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 21, or is identical to SEQ ID NO: 21:

(SEQ ID NO: 21)
5′-TTATCCCTTGCGGTGATAG-3′;

or is an active fragment thereof.

In some embodiments, the cargo polynucleotide comprises a second CRO repressor binding site.

In some embodiments, the at least a first and the second CRO repressor binding sites are upstream of the sequence encoding for the molecule of interest. In some embodiments, the at least a first and the second CRO repressor binding sites are upstream of the promoter region. Thus, in the presence of CRO or CRO-NLS proteins, two individual CRO or CRO-NLS monomers or a CRO or CRO-NLS dimer will substantially bind to the CRO repressor binding sites and prevent the promoter from initiating transcription of the molecule of interest.

In some embodiments, the at least a first and the second CRO repressor binding sites are between the promoter region and the sequence encoding for the molecule of interest of the cargo polynucleotide. Thus, in the presence of CRO or CRO-NLS proteins, two individual CRO or CRO-NLS monomers or a CRO or CRO-NLS dimer will substantially bind to the CRO repressor binding sites and stop the promoter from initiating transcription of the molecule of interest.

In some embodiments, the at least a first CRO repressor binding site is located within the promoter region and the second CRO repressor binding site is located between the promoter region and the sequence encoding for the molecule of interest. In some embodiments, the at least a first CRO repressor binding site is located upstream of the promoter region and the second CRO repressor binding site is located within the promoter region. Thus, in the presence of CRO or CRO-NLS proteins, two individual CRO or CRO-NLS monomers or a CRO or CRO-NLS dimer will substantially bind to the CRO repressor binding sites and prevent the promoter from binding to the promoter region, thus preventing the promoter from initiating transcription of the molecule of interest.

In some embodiments, the second CRO repressor binding site is selected from the group consisting of rightward operators of a bacteriophage and leftward operators of a bacteriophage, as provided for herein.

In some embodiments, the second CRO repressor binding site is selected from the group consisting of rightward operators of bacteriophage λ and leftward operators of bacteriophage λ. In some embodiments, the second CRO repressor binding site is selected from the group consisting of a bacteriophage λ leftward operator 1 (O_{L1}), a bacteriophage λ leftward operator 2 (O_{L2}), a bacteriophage λ leftward operator 3 (O_{L3}), a bacteriophage λ rightward operator 1 (O_{R1}), a bacteriophage λ rightward operator 2 (O_{R2}), and a bacteriophage λ rightward operator 3 (O_{R3}).

In some embodiments, the second CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 16, or is identical to SEQ ID NO: 16, a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 17, or is identical to SEQ ID NO: 17, a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 18, or is identical to SEQ ID NO: 18, a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19, or is identical to SEQ ID NO: 19, a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 20, or is identical to SEQ ID NO: 20, a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 21, or is identical to SEQ ID NO: 21, or is an active fragment of any sequence thereof.

In embodiments comprising two or more CRO repressor binding sites, each binding site may, independently, comprise the nucleic acid sequence of any of SEQ ID NO:16 through SEQ ID NO: 21. Thus, in some embodiments, the at least a first CRO repressor binding site comprises a nucleic acid sequence of SEQ ID NO: 16, SEQ ID NO: 17, SEQ ID NO: 18, SEQ ID NO: 19, SEQ ID NO: 20, or SEQ ID NO: 21, or a sequence substantially similar to SEQ ID NO: 16, SEQ ID NO: 17, SEQ ID NO: 18, SEQ ID NO: 19, SEQ ID NO: 20, or SEQ ID NO: 21, or an active fragment of any sequence thereof, and the second CRO repressor binding site comprises, independently, a nucleic acid sequence of SEQ ID NO: 16, SEQ ID NO: 17, SEQ ID NO: 18, SEQ ID NO: 19, SEQ ID NO: 20, or SEQ ID NO: 21, or a sequence substantially similar to SEQ ID NO: 16, SEQ ID NO: 17, SEQ ID NO: 18, SEQ ID NO: 19, SEQ ID NO: 20, or SEQ ID NO: 21, or an active fragment of any sequence thereof. In some embodiments the sequence identity of the at least a first CRO repressor binding site and the second CRO repressor binding site are the same. In some embodiments the sequence identity of the at least a first CRO repressor binding site and the second CRO repressor binding site are different.

In some embodiments, additional CRO repressor binding sites can be added. In some embodiments, the cargo polynucleotide comprises 1, 2, 3, 4, 5, or more CRO repressor binding sites. In embodiments comprising additional CRO repressor binding sites, each additional binding site can comprise a CRO repressor binding site selected from the group consisting of rightward operators of a bacteriophage and leftward operators of a bacteriophage, as provided for herein. In some embodiments, each additional binding site can comprise, independently a nucleic acid sequence of SEQ ID NO: 16, SEQ ID NO: 17, SEQ ID NO: 18, SEQ ID NO: 19, SEQ ID NO: 20, or SEQ ID NO: 21, or a sequence substantially similar to SEQ ID NO: 16, SEQ ID NO: 17, SEQ ID NO: 18, SEQ ID NO: 19, SEQ ID NO: 20, or SEQ ID NO: 21, or an active fragment of any sequence thereof. In embodiments comprising additional CRO repressor binding sites, the sequence identity of any number of CRO repressor binding sites can be identical and the sequence identity of any number of CRO repressor binding sites can be different. In some embodiments, the cargo polynucleotide comprises at least a first CRO repressor binding site. In some embodiments, the cargo polynucleotide comprises two CRO repressor binding sites.

In further embodiments, the sequence encoding the molecule of interest encodes for one or more of a viral protein, a shRNA, a therapeutic molecule, a tumor antigen, a protein, a nucleic acid molecule, or a combination thereof. In some embodiments, “protein” can mean a membrane bound protein, a cytosolic protein, a nuclear localized protein, a mitochondria localized protein, a chimeric protein, or a fusion protein. In some embodiments, “protein” can mean an enzyme, a nuclear receptor, a transporter, a ribosomal protein, a G-protein coupled receptor, a voltage gated ion channel, a secretory protein, a mitochondria protein, a cytokine, or a chimeric species thereof. In some embodiments, “protein” can mean any polypeptide sequence to be expressed for a function in the cell. In some embodiments, the at least one viral protein can mean any protein encoded by a viral genome. In some embodiments, the at least one viral protein is an essential structural protein. In some embodiments, the at least one viral protein is an essential protein for the proper packaging and construction of a viral particle. In some embodiments, the at least one shRNA can knock down any desired protein target. In some embodiments, the at least one shRNA can knock down any inappropriately over-expressed protein. In some embodiments, the at least one shRNA can knock down any protein harboring a disease causing mutation. In some embodiments, the at least one shRNA can knock down a transcription factor to alter gene expression. In some embodiments, the at least one therapeutic molecule is any molecule used to treat a disease or have a beneficial impact on disease progression.

In some embodiments, the therapeutic molecule is a cytokine, such as IL-2, IL-12, IL-15, and the like.

In some embodiments, a virus is provided. In some embodiments, the virus provided is a recombinant virus. In some embodiments, the recombinant virus is selected from the group consisting of lentivirus, adenovirus, adeno-associated virus, or the like. In some embodiments, the recombinant virus is a recombinant adenovirus.

In some embodiments, the virus comprises a cargo polynucleotide encoding for a molecule of interest. In some embodiments, the cargo polynucleotide further comprises a promoter region operably connected to the molecule of interest. In some embodiments, the cargo polynucleotide encoding a molecule of interest further comprises at least a first CRO repressor binding site. In some embodiments, the virus comprises a cargo polynucleotide comprising a nucleic acid sequence encoding for a molecule of interest, a promoter region operably connected to the molecule of interest, and at least a first CRO repressor binding site.

In some embodiments, the cargo polynucleotide of the virus comprises a 5′ adenoviral ITR and a 3′ adenoviral ITR, wherein the 5′ ITR and 3′ ITR flank the nucleic acid sequence encoding for the molecule of interest, the promoter region operably connected to the molecule of interest, and the least a first CRO repressor binding site.

In some embodiments, the promoter region of the cargo polynucleotide is located upstream of the sequence encoding the molecule of interest.

The at least a first CRO repressor binding site of the cargo polynucleotide can be variably located within the cargo polynucleotide. In some embodiments, the at least a first CRO repressor binding site can be located at a position selected from the list comprising, upstream of the sequence encoding for the molecule of interest, upstream of the promoter region of the cargo polynucleotide, between the promoter region and the sequence encoding for the molecule of interest of the cargo polynucleotide, or within the promoter region of the cargo polynucleotide.

In some embodiments, the at least a first CRO repressor binding site of the cargo polynucleotide of the virus is selected from the group consisting of rightward operators of bacteriophage and leftward operators of bacteriophage, as provided for herein.

In some embodiments, the at least a first CRO repressor binding site of the cargo polynucleotide of the virus is selected from the group consisting of rightward operators of bacteriophage λ and leftward operators of bacteriophage λ. In some embodiments, the at least a first CRO repressor binding site is selected from the group consisting of a bacteriophage λ leftward operator 1 (O_{L1}), a bacteriophage λ leftward operator 2 (O_{L2}), a bacteriophage λ leftward operator 3 (O_{L3}), a bacteriophage λ rightward operator 1 (O_{R1}), a bacteriophage λ rightward operator 2 (O_{R2}), and a bacteriophage λ rightward operator 3 (O_{R3}).

In some embodiments, the at least a first CRO repressor binding site of the cargo polynucleotide of the virus comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 16, or is identical to SEQ ID NO: 16, a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 17, or is identical to SEQ ID NO: 17, a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 18, or is identical to SEQ ID NO: 18, a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19, or is identical to SEQ ID NO: 19 a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 20, or is identical to SEQ ID NO: 20, a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 21, or is identical to SEQ ID NO: 21, or is an active fragment of any sequence thereof.

In some embodiments, the cargo polynucleotide of the virus further comprises a second CRO repressor binding site.

The at least a first CRO repressor binding site and the second CRO repressor binding site of the cargo polynucleotide can be variably located within the cargo polynucleotide. In some embodiments, the at least a first and the second CRO repressor binding sites are both located upstream of the sequence encoding for the molecule of interest. In some embodiments, the at least a first and the second CRO repressor binding sites are both located upstream of the promoter region of the cargo polynucleotide. In some embodiments, the at least a first and the second CRO repressor binding sites are both located between the promoter region of the cargo polynucleotide and the sequence encoding for the molecule of interest. In some embodiments, the at least a first CRO repressor binding site is located within the promoter region of the cargo polynucleotide and the second CRO repressor binding site is located between the promoter region and the sequence encoding for the molecule of interest. In some embodiments, the at least a first CRO repressor binding site is located upstream of the promoter region and the second CRO repressor binding site is located within the promoter region. Thus, in the presence of CRO or CRO-NLS proteins, two individual CRO or CRO-NLS monomers or a CRO or CRO-NLS dimer will substantially bind to the CRO repressor binding sites and prevent the promoter from initiating transcription of the molecule of interest.

In some embodiments, the second CRO repressor binding site of the cargo polynucleotide of the virus is selected from the group consisting of rightward operators of bacteriophage and leftward operators of bacteriophage, as provided for herein.

In some embodiments, the second CRO repressor binding site of the cargo polynucleotide of the virus is selected from the group consisting of rightward operators of bacteriophage λ and leftward operators of bacteriophage λ. In some embodiments, the second CRO repressor binding site is selected from the group consisting of a bacteriophage λ leftward operator 1 (O_{L1}), a bacteriophage λ leftward operator 2 (O_{L2}), a bacteriophage λ leftward operator 3 (O_{L3}), a bacteriophage λ rightward operator 1 (O_{R1}), a bacteriophage λ rightward operator 2 (O_{R2}), and a bacteriophage λ rightward operator 3 (O_{R3}).

In some embodiments, the second CRO repressor binding site of the cargo polynucleotide of the virus comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 16, or is identical to SEQ ID NO: 16, a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 17, or is identical to SEQ ID NO: 17, a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 18, or is identical to SEQ ID NO: 18, a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19, or is identical to SEQ ID NO: 19, a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 20, or is identical to SEQ ID NO: 20, a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 21, or is identical to SEQ ID NO: 21, or is an active fragment of any sequence thereof.

In embodiments comprising two or more CRO repressor binding sites, each binding site may, independently, comprise the nucleic acid sequence of any of SEQ ID NO: 16 through SEQ ID NO: 21. Thus, in some embodiments, the at least a first CRO repressor binding site comprises a nucleic acid sequence of SEQ ID NO: 16, SEQ ID NO: 17, SEQ ID NO: 18, SEQ ID NO: 19, SEQ ID NO: 20, or SEQ ID NO: 21, or a sequence substantially similar to SEQ ID NO: 16, SEQ ID NO: 17, SEQ ID NO: 18, SEQ ID NO: 19, SEQ ID NO: 20, or SEQ ID NO: 21, or an active fragment of any sequence thereof, and the second CRO repressor binding site comprises, independently, a nucleic acid sequence of SEQ ID NO: 16, SEQ ID NO: 17, SEQ ID NO: 18, SEQ ID NO: 19, SEQ ID NO: 20, or SEQ ID NO: 21, or a sequence substantially similar to SEQ ID NO: 16, SEQ ID NO: 17, SEQ ID NO: 18, SEQ ID NO: 19, SEQ ID NO: 20, or SEQ ID NO: 21, or an active fragment of any sequence thereof. In some embodiments the sequence identity of the at least a first CRO repressor binding site and the second CRO repressor binding site are the same. In some embodiments the sequence identity of the at least a first CRO repressor binding site and the second CRO repressor binding site are different.

In further embodiments, additional CRO repressor binding sites can be added. In some embodiments, the cargo polynucleotide comprises 1, 2, 3, 4, 5, or more CRO repressor binding sites. In embodiments comprising additional CRO repressor binding sites, each additional binding site can comprise a CRO repressor binding site selected from the group consisting of rightward operators of a bacteriophage and leftward operators of a bacteriophage, as provided for herein. In some embodiments, each additional binding site can comprise, independently a nucleic acid sequence of SEQ ID NO: 16, SEQ ID NO: 17, SEQ ID NO: 18, SEQ ID NO: 19, SEQ ID NO: 20, or SEQ ID NO: 21, or a sequence substantially similar to SEQ ID NO: 16, SEQ ID NO: 17, SEQ ID NO: 18, SEQ ID NO: 19, SEQ ID NO: 20, or SEQ ID NO: 21, or an active fragment of any sequence thereof. In embodiments comprising additional CRO repressor binding sites, the sequence identity of any number of CRO repressor binding sites can be identical and the sequence identity of any number of CRO repressor binding sites can be different. In some embodiments, the cargo polynucleotide comprises at least a first CRO repressor binding site. In some embodiments, the cargo polynucleotide comprises two CRO repressor binding sites.

In some embodiments, the sequence encoding the molecule of interest of the cargo polynucleotide of the virus encodes for one or more of a viral protein, a shRNA, a therapeutic molecule, a tumor antigen, a protein, a nucleic acid molecule, or a combination thereof.

In some embodiments, the therapeutic molecule is a cytokine, such as IL-2, IL-12, IL-15, antigen, tumor antigen, protein, viral antigen, and the like.

In some embodiments, the virus provided is a recombinant virus. In some embodiments, the recombinant virus is selected from the group consisting of lentivirus, adenovirus, or adeno-associated virus. In some embodiments, the recombinant virus is a recombinant adenovirus. In some embodiments, the recombinant virus is replication-incompetent or replication competent.

In some embodiments, the recombinant adenovirus is replication-incompetent or replication competent.

In some embodiments, the replication-incompetent recombinant virus further comprises a defective or modified E1 gene, E3 gene, E4 gene, E4 promoter, hexon gene, penton gene, fiber gene, or combination thereof. In some embodiments, the replication-incompetent recombinant virus comprises a defective or modified E1 gene.

In some embodiments a host cell is provided. In some embodiments, the host cell contains a competent E1 gene, E3 gene, E4 gene, E4 promoter, hexon gene, penton gene, fiber gene or a combination thereof to complement any defective or modified gene in the recombinant virus. In some embodiments, the host cell contains a competent E1 gene to complement the defective or modified E1 gene in the recombinant virus. In some embodiments, the competent E1 gene, E3 gene, E4 gene, E4 promoter, hexon gene, penton gene, fiber gene or a combination thereof are provided to the host cell via contacting the host cell transiently. In some embodiments, the host cell contains, within its genome, the competent E1 gene, E3 gene, E4 gene, E4 promoter, hexon gene, penton gene, fiber gene or a combination thereof. In a further embodiment, a plasmid is provided. In some embodiments, the plasmid comprises any of the CRO-NLS polynucleotide molecules described herein.

In a further embodiment, a cell is provided. In some embodiments, the cell comprises any of the CRO-NLS polynucleotide molecules described herein. In some embodiments, the cell comprises any of the plasmids comprising any of the CRO-NLS polynucleotide molecules described herein.

In some embodiments, a vector is provided comprising both the first polynucleotide molecule and the cargo polynucleotide molecule. In some embodiments, the vector is a plasmid. In some embodiments, the vector is a virus. In some embodiments, expression of the first polynucleotide molecule is under the control of a first promoter region and expression of the cargo polynucleotide molecule is under the control of a second promoter region. In some embodiments, expression of both the first polynucleotide molecule and the cargo polynucleotide molecule are under the control of the same promoter. In some embodiments, the sequence of the first polynucleotide molecule and the sequence of the cargo polynucleotide molecule are linked by an additional nucleic acid sequence. In some embodiments, the sequence of the first polynucleotide molecule and the sequence of the cargo polynucleotide molecule are linked by an internal ribosome entry site (IRES) sequence. The identity of the IRES sequence can be any IRES sequence known in the art. In some embodiments, the sequence of the first polynucleotide molecule and the sequence of the cargo polynucleotide molecule are linked by a self-cleaving peptide site. In some embodiments, the self-cleaving peptide site is a 2A site. In some embodiments, the 2A site is selected from the group comprising T2A, P2A, E2A, and F2A. In some embodiments, a plasmid is provided comprising the vector containing the first polynucleotide molecule and the cargo polynucleotide molecule.

In some embodiments, a polypeptide is provided comprising a nuclear localization signal linked to a heterologous molecule. In some embodiments, the NLS comprises any amino acid sequence known to target the heterologous molecule to the nucleus. In some embodiments, the NLS comprises one or more NLS sequences that are known to target the heterologous molecule to the nucleus. In some embodiments, the NLS comprises at least two NLS sequences that are known to target the heterologous molecule to the nucleus. In some embodiments, the at least two NLS sequences form a contiguous sequence with no linking sequence separating the two NLS sequences. In some embodiments, the at least two NLS sequences are joined by a linking sequence.

In some embodiments, the NLS of the polypeptide comprises a formula NLS₁-Xₙ-NLS₂, wherein NLS₁ and NLS₂ are each, independently, any nuclear localization signal known to target a heterologous molecule to the nucleus, and Xₙ is a peptide linker. In some embodiments, wherein X is any amino acid. In some embodiments, Xₙ comprises 1-20 amino acid residues. In some embodiments, the linker comprises 20-30, 20-40, or more than 40 amino acid residues. In some embodiments, the linker comprises 1, 2, 3, 4, 5, 6, 7, 8, 9, 10, 11, 12, 13, 14, 15, 16, 17, 18, 19, or 20 amino acid residues in length. In some embodiments the linker is absent. In some embodiments, the Xₙ peptide linker is as provided for herein.

In some embodiments, NLS₁ is selected from the group comprising SEQ ID NO: 3, SEQ ID NO: 4, or a combination thereof. In some embodiments, NLS₂ is selected from the group comprising SEQ ID NO: 3, SEQ ID NO: 4, or a combination thereof. In some embodiments, NLS₁ and NLS₂ comprise the same amino acid sequence. In some embodiments, NLS₁ and NLS₂ comprise different amino acid sequences.

In some embodiments, the NLS of the polypeptide comprises a formula of NLS₁-Xₙ-NLS₂, wherein NLS₁ comprises an amino acid sequence of SEQ ID NO: 3, NLS₂ comprises an amino acid sequence of SEQ ID NO:4, X is any amino acid, and n is 9. In some embodiments, Xₙ comprises an amino acid sequence of ATESQDTGP (SEQ ID NO: 33). In some embodiments, the NLS of the polypeptide is a synthetic bipartite NLS and comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 5, or comprises SEQ ID NO: 5, or an amino acid sequence substantially similar to SEQ ID NO: 5, or an active fragment thereof.

In some embodiments, the heterologous molecule is a polypeptide, such that a fusion polypeptide is formed between the NLS and the polypeptide. In some embodiments, the heterologous molecule comprises a nucleic acid molecule.

In some embodiments, the NLS and the heterologous molecule of the polypeptide form a contiguous molecule such that no additional linker sequence is added. In some embodiments, the polypeptide further comprises a linker sequence linking the NLS and the heterologous molecule. In some embodiments, the linker sequence is a nucleotide sequence. In some embodiments, the linker sequence is a peptide linker sequence, such as a glycine/serine (GS) linker. In some embodiments, the GS linker comprises an amino acid sequence including but not limited to (GGGGS)ₙ(SEQ ID NO: 22), (GGGSS)ₙ(SEQ ID NO: 23), (GSGSG)ₙ(SEQ ID NO: 24), or (GSSG)ₙ(SEQ ID NO: 25), wherein each n is, independently, from 1 and 5, or a combination thereof. In some embodiments, n is 1. In some embodiments, n is 2. In some embodiments, n is 3. In some embodiments, n is 4. In some embodiments, n is 5. In some embodiments the linker is a cleavable linker. In some embodiments, the cleavable linker comprises a sequence of GPGSKLKSGFGGPGSRFRSGPG (SEQ ID NO: 1). In some embodiments, the linker is a non-cleavable linker. In some embodiments the non-cleavable linker comprises a sequence of AAGGTGGGSGGGTGGS (SEQ ID NO: 2). In some embodiments, the linker encoded by the polynucleotide is a peptide linker including, but not limited to SEQ ID NO: 22, SEQ ID NO: 23, SEQ ID NO: 24, SEQ ID NO: 25, SEQ ID NO: 1, or SEQ ID NO: 2, or any combination thereof, wherein each n is, independently, 1-5.

In some embodiments, the peptide linker comprises an amino acid sequence including, but not limited to, MXGXG, IXGXG, LXGXG, MXGGX, IXGGX, LXGGX, or any combination thereof, or any repetition thereof, wherein each X can be, independently, any amino acid. In some embodiments, the peptide linker is a peptide linker including, but not limited to, GPGPG (SEQ ID NO: 32), or any repetition thereof. In some embodiments, the peptide linker is a peptide linker including, but not limited to, GPGPG (SEQ ID NO: 32).

In some embodiments, the NLS sequence is located within the heterologous molecule. For example, the NLS sequence could be incorporated into an intrinsically disordered domain of a polypeptide heterologous molecule. Said intrinsically disordered domain could comprise, as a non-limiting example, the intra-helical domain of a transmembrane protein comprised of multiple alpha-helical domains. The intrinsically disordered domain can comprise any intrinsically disordered domain such that the NLS is available to target its cognate binding partner and translocate the polypeptide into the nucleus.

In some embodiments, the molecule comprises a second heterologous molecule. The second heterologous molecule can be selected from the group comprising a polypeptide molecule or a nucleic acid molecule. Thus, the molecule comprising an NLS, a first heterologous molecule, and a second heterologous molecule could comprise an NLS and two nucleic acid molecules, an NLS and two polypeptide molecules, or an NLS, one nucleic acid molecule and one polypeptide molecule.

The location of the NLS can be variable such that any location may be utilized as long as it does not hinder or inhibit the native function of the NLS. Thus, the NLS could be located at the N terminus of the polypeptide molecule, the C terminus of the polypeptide molecule, between the at least a first heterologous molecule and a second heterologous molecule, within the at least a first heterologous molecule, within the second heterologous molecule, etc.

In some embodiments, a polynucleotide molecule is provided comprising a promoter region and a polynucleotide sequence encoding for the polypeptide molecule comprising an NLS and an at least a first heterologous molecule. In some embodiments, the polynucleotide molecule further encodes for a second heterologous molecule.

### Pharmaceutical Compositions

In another aspect, the present embodiments provide compositions, e.g., pharmaceutically acceptable compositions, which include a polypeptide as provided for herein or a nucleic acid molecule encoding the same, which can be, for example, be formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, local, topical, spinal or epidermal administration (e.g. by injection or infusion). In some embodiments, the pharmaceutical composition comprises a vector comprising a nucleic acid molecule encoding a polypeptide as provided for herein. In some embodiments, the nucleic acid molecule is a DNA molecule or a RNA molecule. In some embodiments, the vector is a virus, such as those provided for herein

The compositions may be in a variety of forms. These include, for example, liquid, semi-solid and solid dosage forms, such as liquid solutions (e.g., injectable and infusible solutions), dispersions or suspensions, liposomes and suppositories. The form depends on the intended mode of administration and therapeutic application. Typical compositions are in the form of injectable or infusible solutions. In an embodiment the mode of administration is parenteral (e.g., intravenous, subcutaneous, intraperitoneal, intramuscular). In an embodiment, the therapeutic molecule is administered by intravenous infusion or injection. In some embodiments, the therapeutic molecule is administered by intramuscular or subcutaneous injection. In some embodiments, the therapeutic molecule is administered locally, e.g., by injection, or topical application, to a target site. For example, the pharmaceutical compositions can be lyophilized and reconstituted for use prior to administration to the patient.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high therapeutic molecule concentration. Sterile injectable solutions can be prepared by incorporating the active compound (i.e., therapeutic molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation can be vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In some embodiments, the pharmaceutical composition can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer composition by other than parenteral administration, it may be necessary to coat the composition with, or co-administer the compound with, a material to prevent its inactivation. Therapeutic compositions can also be administered with medical devices known in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a therapeutic compound is 0.1-30 mg/kg, more preferably 1-25 mg/kg. Dosages and therapeutic regimens of the therapeutic compound can be determined by a skilled artisan. In certain embodiments, the therapeutic compound is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks, or, in some embodiments, the dosing schedule can be, once every month, every 2 months, every 3 months, or every 6 months. In one embodiment, the therapeutic compound is administered at a dose from about 10 to 20 mg/kg every other week. The therapeutic compound can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the infusion rate of about 110 to 130 mg/m² achieves a level of about 3 mg/kg. In other embodiments, the therapeutic compound can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the therapeutic compound is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions may include a “therapeutically effective amount” or a “prophylactically effective amount” of the compositions, polypeptides, or nucleic acid molecules encoding the same. A “therapeutically effective amount” refers to an amount effective, at dosages and for periods of time necessary, to achieve the desired therapeutic result. A therapeutically effective amount of a therapeutic molecule may vary according to factors such as the disease state, age, sex, and weight of the individual, and the ability of the therapeutic compound to elicit a desired response in the individual. A therapeutically effective amount is also one in which any toxic or detrimental effects of a therapeutic molecule t is outweighed by the therapeutically beneficial effects. A “therapeutically effective dosage” preferably inhibits a measurable parameter, e.g., tumor growth, by at least about 20%, by at least about 40%, by at least about 60%, and by at least about 80% relative to untreated subjects. The ability of a compound to inhibit a measurable parameter, e.g., tumor growth, can be evaluated in an animal model system predictive of efficacy in tumor growth. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount can be less than the therapeutically effective amount.

Also provided herein are kits comprising compositions as described herein. The kit can include one or more other elements including: instructions for use; other reagents, e.g., a label, a therapeutic agent, or an agent useful for chelating, or otherwise coupling, a therapeutic molecule to a label or other therapeutic agent, or a radioprotective composition; devices or other materials for preparing the therapeutic molecule for administration; pharmaceutically acceptable carriers; and devices or other materials for administration to a subject.

### Combinations

The compositions provided herein can also be administered in conjunction with other agents useful for treating the condition with which the patient is suffering from. Examples of such agents include both proteinaceous and non-proteinaceous drugs. When multiple therapeutics are co-administered, dosages may be adjusted accordingly, as is recognized in the pertinent art. “Co-administration” and combination therapy are not limited to simultaneous administration, but also include treatment regimens in which compositions provided for herein are administered at least once during a course of treatment that involves administering at least one other therapeutic agent to the patient.

### Methods

In certain embodiments, methods of preparing and using the compositions described herein are provided.

In some embodiments, a method of manufacturing the first polynucleotide molecule is provided. In some embodiments, the method comprises (a) providing a recombinant cell that comprises the first polynucleotide molecule or a vector or plasmid containing the first polynucleotide molecule, (b) growing the recombinant cell under conditions for production of the first polynucleotide molecule; and (c) isolating the first polynucleotide molecule from the recombinant cell. The cell can be any cell that will produce the first polynucleotide molecule. In some embodiments, the cell is a strain of Escherichia coli. In some embodiments, the cell is a mammalian cell. Methods of isolating polynucleotide molecules from recombinant cells are well known in the art, and the first polynucleotide molecule may be isolated from the recombinant cell via any such method.

In some embodiments, a method of manufacturing the cargo polynucleotide molecule is provided. In some embodiments, the method comprises (a) providing a recombinant cell that comprises the cargo polynucleotide molecule or a vector or plasmid containing the cargo polynucleotide molecule, (b) growing the recombinant cell under conditions for production of the cargo polynucleotide molecule; and (c) isolating the cargo polynucleotide molecule from the recombinant cell. The cell can be any cell that will produce the second polynucleotide molecule.

In some embodiments, the cell is a strain of Escherichia coli. In some embodiments, the cell is a mammalian cell. Methods of isolating polynucleotide molecules from recombinant cells are well known in the art, and the cargo polynucleotide molecule may be isolated from the recombinant cell via any such method.

In some embodiments, a method of manufacturing a vector or plasmid provided for herein is provided. In some embodiments, the vector or plasmid contains only the first polynucleotide molecule. In some embodiments, the vector or plasmid contains only the cargo polynucleotide molecule. In some embodiments, the vector or plasmid contains both the first polynucleotide molecule and the cargo polynucleotide molecule as provided for herein. In some embodiments, the method comprises (a) providing a recombinant cell that comprises a vector or plasmid containing the first polynucleotide molecule, the cargo polynucleotide molecule, or both the first polynucleotide molecule and the cargo polynucleotide molecule as provided for herein, (b) growing the recombinant cell under conditions for production of the vector or plasmid; and (c) isolating the vector or plasmid from the recombinant cell. As highlighted above, the cell can be any cell that will produce the vector or plasmid. In some embodiments, the cell is a strain of Escherichia coli. In some embodiments, the cell is a mammalian cell. Methods of isolating vectors or plasmids from recombinant cells are well known in the art, and the vector or plasmid may be isolated from the recombinant cell via any such method.

In some embodiments, a method is provided for making a virus comprising the cargo polynucleotide as described herein. In some embodiments, the method comprises of: contacting a host cell with a vector comprising at least any of the cargo polynucleotides as described herein, and culturing the cell under conditions to produce the virus. In some embodiments, the vector further comprises the necessary components to construct the virus. In some embodiments, the method further comprises providing a separate vector containing the necessary components to construct the virus. In some embodiments, the host cell further comprises, within its genome, the necessary components to construct the virus. In some embodiments, the virus produced by the method is an adenovirus. In some embodiments, the virus is replication incompetent or replication competent.

In some embodiments, a method is provided for producing a plurality of recombinant therapeutic viruses. In some embodiments, the method comprises (a) providing a host cell, (b) delivering to the host cell a first polynucleotide molecule encoding a CRO-NLS protein as described herein, (c) delivering to the host cell a cargo polynucleotide molecule comprising at least one promoter region, at least a first CRO repressor binding site, and a sequence encoding for a viral payload, (d) culturing the host cell under conditions that reduce the expression of the viral payload by binding the CRO repressor protein to the at least a first CRO repressor binding site, wherein culturing the host cell under said conditions prevents cellular toxicity associated with the viral payload and produces a plurality of recombinant viruses.

In some embodiments, the host cell is genetically modified to express the CRO-NLS repressor protein. In some embodiments, the CRO repressor protein comprises an amino acid sequence selected from the group consisting of SEQ ID NO: 8, SEQ ID NO: 10, SEQ ID NO: 12, or SEQ ID NO: 14. A person of ordinary skill will recognize that a given protein will tolerate a certain degree of substitutions of amino acids for alternate amino acids without negatively affecting the native function of the protein. In some cases, the substitution replaces an amino acid for one with similar properties (for example, GLU for ASP). In some cases, the substitution replaces an amino acid for one that may have dissimilar properties (for example, ARG for ALA). Therefore, in some embodiments, the CRO repressor protein comprises an amino acid sequence that is similar or substantially similar to an amino acid sequence selected from the group consisting of SEQ ID NO: 8, SEQ ID NO: 10, SEQ ID NO: 12, or SEQ ID NO: 14.

In some embodiments, the cargo polynucleotide molecule comprising at least one promoter region, at least a first CRO repressor binding site, and a sequence encoding for a viral payload is a viral vector. In some embodiments, the viral vector further encodes for a recombinant virus. The recombinant virus may be a recombinant lentivirus, recombinant adenovirus, or a recombinant adeno-associated virus. In some embodiments, the recombinant virus is a recombinant adenovirus. In some embodiments, the recombinant virus is replication incompetent or replication competent. In some embodiments, the recombinant virus is replication incompetent and further comprises a defective or modified E1 gene, E3 gene, E4 gene, E4 promoter, hexon gene, penton gene, fiber gene, or a combination thereof. In some embodiments, the virus is replication-incompetent and further comprises a defective or modified E1 gene.

In some embodiments, the host cell provided by the method contains a competent E1 gene, E3 gene, E4 gene, E4 promoter, hexon gene, penton gene, fiber gene or a combination thereof to complement any defective or modified gene in the recombinant virus. In some embodiments, the competent E1 gene, E3 gene, E4 gene, E4 promoter, hexon gene, penton gene, fiber gene or a combination thereof are provided to the host cell transiently. In some embodiments, the host cell provided by the method contains, within its genome, a competent E1 gene, E3 gene, E4 gene, E4 promoter, hexon gene, penton gene, fiber gene or a combination thereof to complement any defective or modified gene in the recombinant virus. In some embodiments, the host cell contains, within its genome, a competent E1 gene to complement the defective or modified E1 gene in the recombinant virus.

In some embodiments, a method to control the expression of a cargo of interest is provided. In some embodiments, the method comprises (a) providing a host cell comprising a polynucleotide encoding any of the CRO-NLS repressor proteins described herein or comprising a polypeptide comprising any of the CRO-NLS repressor proteins described herein, and (b) contacting the host cell with a cargo polynucleotide comprising a nucleic acid sequence encoding for a molecule of interest, a promoter region operably connected to the molecule of interest, and at least a first CRO repressor binging site, wherein the expression of the molecule of interest is controlled by the binding of the CRO-NLS protein.

In some embodiments, the host cell is genetically modified to express the CRO-NLS repressor protein. In some embodiments, controlling the expression of a molecule of interest comprises reducing or preventing the expression of the molecule of interest, and the binding of the CRO-NLS repressor protein to the at least a first CRO repressor binding site reduces or prevents the expression of the molecule of interest. In some embodiments, the CRO-NLS repressor protein comprises an amino acid sequence selected from the group consisting of SEQ ID NO: 8, SEQ ID NO: 10, SEQ ID NO: 12, or SEQ ID NO: 14. A person of ordinary skill will recognize that a given protein will tolerate a certain degree of substitutions of amino acids for alternate amino acids without negatively affecting the native function of the protein. In some cases, the substitution replaces an amino acid for one with similar properties (for example, GLU for ASP). In some cases, the substitution replaces an amino acid for one that may have dissimilar properties (for example, ARG for ALA). Therefore, in some embodiments, the CRO repressor protein comprises an amino acid sequence that is similar or substantially similar to an amino acid sequence selected from the group consisting of SEQ ID NO: 8, SEQ ID NO: 10, SEQ ID NO: 12, or SEQ ID NO: 14.

In some embodiments, the molecule of interest of the provided method is toxic to the host cell. In some embodiments, the molecule of interest is one or more of a viral protein, a shRNA, a therapeutic molecule, a tumor antigen, a protein, a nucleic acid molecule, or a combination thereof.

In some embodiments, a method is provided for delivering a molecule of interest to a subject. In some embodiments, the method comprises administering a virus to the subject, wherein the virus comprises a cargo polynucleotide comprising at least one promoter region, at least a first CRO repressor binding site, and a sequence encoding a molecule of interest.

In some embodiments, a method is provided for inducing an immune response in a subject against a molecule of interest. In some embodiments, the method comprises administering a virus to the subject, wherein the virus comprises a cargo polynucleotide comprising at least one promoter region, at least a first CRO repressor binding site, and a sequence encoding a molecule of interest, and wherein the molecule of interest is a viral protein or a tumor antigen. Thus, expression of the viral protein or the tumor antigen by the target cell in the subject will induce an immune response targeting the viral protein or tumor antigen.

In some embodiments, a method of treating a disease or disorder is provided. In some embodiments, the method comprises administering a virus to the subject, wherein the virus comprises a cargo polynucleotide comprising at least one promoter region, at least a first CRO repressor binding site, and a sequence encoding a molecule of interest, wherein the molecule of interest is useful in the treatment of a disease or disorder.

In some embodiments, the method comprises (a) providing a host cell, (b) delivering to the host cell a first polynucleotide molecule encoding a CRO-NLS protein as described herein, (c) delivering to the host cell a cargo polynucleotide molecule comprising at least one promoter region, at least a first CRO repressor binding site, and a sequence encoding for a molecule of interest, (d) culturing the host cell under conditions that reduce the expression of the molecule of interest by binding the CRO repressor protein to the at least a first CRO repressor binding site, wherein culturing the host cell under said conditions prevents cellular toxicity associated with the molecule of interest and produces a plurality of recombinant viruses (e) harvesting the viral particles, and (f) administering to the subject a pharmaceutical composition comprising the viral particles, wherein the molecule of interest is useful in the treatment of a disease or disorder.

In some embodiments, a kit is provided for controlling the expression of a target of interest in a cell. In some embodiments, the kit comprises (a) an expression vector comprising at least on promoter region, at least a first CRO repressor binding site, a sequence encoding for the cargo of interest, and a multiple cloning site, wherein both the at least one promoter and at least a first CRO repressor binding sites are upstream of the multiple cloning site, and (b) a host cell that is genetically modified to express a chimeric CRO repressor protein comprising at least one chimeric CRO domain and a nuclear localization sequence domain. In some embodiments, the CRO repressor protein comprises an amino acid sequence selected from the group consisting of SEQ ID NO: 8, SEQ ID NO: 10, SEQ ID NO: 12, or SEQ ID NO: 14. A person of ordinary skill will recognize that a given protein will tolerate a certain degree of substitutions of amino acids for alternate amino acids without negatively affecting the native function of the protein. In some cases, the substitution replaces an amino acid for one with similar properties (for example, GLU for ASP). In some cases, the substitution replaces an amino acid for one that may have dissimilar properties (for example, ARG for ALA). Therefore, in some embodiments, the CRO repressor protein comprises an amino acid sequence that is similar or substantially similar to an amino acid sequence selected from the group consisting of SEQ ID NO: 8, SEQ ID NO: 10, SEQ ID NO: 12, or SEQ ID NO: 14.

In some embodiments, a method is provided for directing a heterologous molecule of interest to the nucleus of a cell. In some embodiments, the method comprises providing a polypeptide molecule comprising an NLS sequence and a heterologous molecule of interest, contacting a cell with the polypeptide molecule. The NLS can direct the heterologous molecule of interest to the nucleus. In some embodiments, the NLS sequence is a synthetic bipartite NLS and comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 5, or comprises SEQ ID NO: 5, or an amino acid sequence substantially similar to SEQ ID NO: 5, or an active fragment thereof. In some embodiments the heterologous molecule of interest is a polypeptide. In some embodiments, the heterologous molecule of interest is a polynucleotide. In some embodiments, the polypeptide molecule further comprises a second heterologous molecule of interest.

In some embodiments, the method comprises providing a polynucleotide molecule encoding for a polypeptide molecule comprising an NLS sequence and a heterologous molecule of interest, contacting a cell with the polynucleotide molecule, wherein the NLS sequence will direct the heterologous molecule of interest to the nucleus. In some embodiments, the NLS sequence is a synthetic bipartite NLS and comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 5, or comprises SEQ ID NO: 5, or an amino acid sequence substantially similar to SEQ ID NO: 5, or an active fragment thereof.

In some embodiments, the method comprises providing a vector encoding for a polypeptide molecule comprising an NLS sequence and a heterologous molecule of interest, contacting a cell with the vector, wherein the NLS sequence will direct the heterologous molecule of interest to the nucleus. In some embodiments, the NLS sequence is a synthetic bipartite NLS and comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 5, or comprises SEQ ID NO: 5, or an amino acid sequence substantially similar to SEQ ID NO: 5, or an active fragment thereof. In some embodiments the vector is a plasmid. In some embodiments, the vector is a virus.

“Treatment” of any disease mentioned herein encompasses an alleviation of at least one symptom of the disease, a reduction in the severity of the disease, or the delay or prevention of disease progression to more serious symptoms that may, in some cases, accompany the disease or to at least one other disease. Treatment need not mean that the disease is totally cured. A useful therapeutic agent needs only to reduce the severity of a disease, reduce the severity of symptom(s) associated with the disease or its treatment, or delay the onset of more serious symptoms or a more serious disease that can occur with some frequency following the treated condition. For example, if the disease is a tumor, the composition may reduce the growth or spread of the tumor, or the tumors effect on the tissue in which it is present. A patient's condition can be assessed by standard techniques. Suitable procedures vary according to the patient's condition and symptoms.

In some embodiments, the compositions provided for herein can be used to modifying an immune response in a patient. In some embodiments, the methods comprise administering to the patient a vector comprising a nucleic acid molecule encoding for a polypeptide as provided for herein. In some embodiments, the immune response is an activated immune response, such as in activating NK and/or CD8+ T cells.

In some embodiments, the compositions provided for herein can be used to treat cancer in a subject (patient). In some embodiments, the methods comprise administering to the patient a vector comprising a nucleic acid molecule encoding for a polypeptide as provided for herein. In some embodiments, the cancer is lymphoma, leukemia, nasopharyngeal, gastric, cervical, hepatocellular, polyoma, anal, head and neck tumor. In some embodiments, the tumor is a lung cancer tumor. In some embodiments, the tumor is benign and metastatic forms of cancer, for example, ovarian cancer (e.g. ovarian carcinoma), reproductive cancers (breast, cervical, testicular, uterine, and placental cancers), lung cancer, gastric cancer, hepatic cancer, pancreatic cancer, bile duct cancer, cancer of the urinary bladder, kidney cancer, colon cancer, small bowel cancer, skin cancer, brain cancer, head and neck cancer, sarcoma, and germ cell tumors, among others.

In some embodiments, diseases that can be treated with the compositions provided for herein also include myelodysplastic syndrome (MDS) or acute myeloid leukemia (AML). In some embodiments, the subject has MDS including Fanconi Anemia, refractory anemia, refractory neutropenia, refractory thrombocytopenia, refractory anemia with ringed sideroblasts (RARS), refractory cytopenia with multilineage dysplasia (RCMD), refractory anemia with multilineage dysplasia and ringed sideroblasts (RCMD-RS), refractory anemia with excess blasts I and II (RAEB), myelodysplastic syndrome, unclassified (MDS-U), MDS associated with isolated del (5q)-syndrome, chronic myelomonocytic leukemia (CMML), juvenile myelomonocytic leukemia (JMML), refractory cytopenia of childhood, or a combination thereof. In some embodiments, the subject has AML including AML with recurrent genetic abnormalities (AML with translocation between chromosomes 8 and 21, AML with translocation or inversion in chromosome 16, AML with translocation between chromosomes 9 and 11, APL (M3) with translocation between chromosomes 15 and 17, AML with translocation between chromosomes 6 and 9, AML with translocation or inversion in chromosome 3), AML (megakaryoblastic) with a translocation between chromosomes 1 and 22, AML with myelodysplasia-related changes, AML related to previous chemotherapy or radiation (alkylating agent-related AML, topoisomerase II inhibitor-related AML), AML not otherwise categorized (AML minimally differentiated (M0), AML with minimal maturation (M1), AML with maturation (M2), acute myelomonocytic leukemia (M4), acute monocytic leukemia (M5), acute erythroid leukemia (M6), acute megakaryoblastic leukemia (M7), acute basophilic leukemia, acute panmyelosis with fibrosis), myeloid sarcoma (also known as granulocytic sarcoma, chloroma or extramedullary myeloblastoma), undifferentiated and biphenotypic acute leukemias (also known as mixed phenotype acute leukemias), or a combination thereof. In some embodiments, administration of the compositions provided for herein to a subject decreases the incidence of one or more symptoms associated with MDS or AML or decreases one or more markers of viability of MDS or AML cells. In some embodiments, the one or more symptoms associated with MDS or AML include decreasing marrow failure, immune dysfunction, transformation to overt leukemia, or a combination thereof in the subject, or wherein the marker of viability of MDS or AML cells includes survival over time, proliferation, growth, migration, formation of colonies, chromatic assembly, DNA binding, RNA metabolism, cell migration, cell adhesion, inflammation, or a combination thereof.

In some embodiments, the tumor is also treated with a PD-1 inhibitor, such as a PD-1 antagonist, such as PD-1 antagonist antibodies.

In some embodiments, the compositions provided for herein can be used to treat a viral infection, a bacterial infection, or a fungal infection in a subject (patient). In some embodiments, the methods comprise administering a pharmaceutical composition comprising the polypeptides provided herein or a nucleic acid molecule encoding the same as provided for herein to the subject. In some embodiments, the subject is a subject in need thereof. Any of the above-described can be administered in the form of a compositions (e.g. pharmaceutical compositions) that are described herein.

To treat the disease of interest, the compositions comprising therapeutic molecules described herein can be administered by any appropriate method including, but not limited to, parenteral, topical, oral, nasal, vaginal, rectal, or pulmonary (by inhalation) administration. If injected, the composition(s) can be administered intra-articularly, intravenously, intraarterially, intramuscularly, intraperitoneally, or subcutaneously by bolus injection or continuous infusion. Localized administration, that is, at the site of disease, is contemplated, as are transdermal delivery and sustained release from implants, skin patches, or suppositories. Delivery by inhalation includes, for example, nasal or oral inhalation, use of a nebulizer, inhalation in aerosol form, and the like. Administration via a suppository inserted into a body cavity can be accomplished, for example, by inserting a solid form of the composition in a chosen body cavity and allowing it to dissolve. Other alternatives include eyedrops, oral preparations such as pills, lozenges, syrups, and chewing gum, and topical preparations such as lotions, gels, sprays, and ointments. In most cases, therapeutic molecules that are polypeptides can be administered topically or by injection or inhalation.

In the performance of the methods of treatment, the therapeutic molecules described above can be administered as described herein and above. For example, the composition can be administered at any dosage, frequency, and duration that can be effective to treat the condition being treated. The dosage depends on the molecular nature of the therapeutic molecule and the nature of the disorder being treated. Treatment may be continued as long as necessary to achieve the desired results. Therapeutic molecules can be administered as a single dosage or as a series of dosages given periodically, including multiple times per day, daily, every other day, twice a week, three times per week, weekly, every other week, and monthly dosages, among other possible dosage regimens. The periodicity of treatment may or may not be constant throughout the duration of the treatment. For example, treatment may initially occur at weekly intervals and later occur every other week. Treatments having durations of days, weeks, months, or years are encompassed by the embodiments provided for herein. Treatment may be discontinued and then restarted. Maintenance doses may or may not be administered after an initial treatment.

Dosage may be measured as milligrams per kilogram of body weight (mg/kg) or as milligrams per square meter of skin surface (mg/m²) or as a fixed dose, irrespective of height or weight. All of these are standard dosage units in the art. A person's skin surface area is calculated from her height and weight using a standard formula.

### Enumerated Embodiments

In some embodiments, the following embodiments are provided:

1. A polynucleotide comprising at least one promoter region operably connected to a nucleotide sequence encoding for a CRO repressor protein operably connected to a nuclear localization signal (“CRO-NLS protein”).

2. The polynucleotide of embodiment 1, wherein the nucleotide sequence encoding for the CRO-NLS protein encodes a CRO repressor protein linked to the NLS by a linker.

3. The polynucleotide of embodiment 2, wherein the linker encoded by the polynucleotide is a peptide linker, such as a glycine/serine linker, including, but not limited to, (GGGGS)ₙ(SEQ ID NO: 22), (GGGSS)ₙ(SEQ ID NO: 23), (GSGSG)ₙ(SEQ ID NO: 24), (GSSG)ₙ(SEQ ID NO: 25), GPGSKLKSGFGGPGSRFRSGPG (SEQ ID NO: 1), or AAGGTGGGSGGGTGGS (SEQ ID NO: 2), or any combination thereof, wherein each n is, independently, 1-5.

4. The polynucleotide of embodiment 2, wherein the linker encoded by the polynucleotide is a peptide linker, including, but not limited to, MXGXG, IXGXG, LXGXG, MXGGX, IXGGX, LXGGX, wherein each X is any amino acid.

5. The polynucleotide of embodiment 2, wherein the linker encoded by the polynucleotide is a peptide linker, including, but not limited to, GPGPG (SEQ ID NO: 32).

6. The polynucleotide of any one of embodiments 1-5, wherein CRO-NLS protein encoded by the nucleotide sequence comprises a monomer or dimer of a CRO repressor protein.

7. The polynucleotide of any one of embodiments 1-6, wherein the CRO-NLS protein comprises a polypeptide having a formula of: X₂-L₂-X₃, wherein:
X₂ is a CRO repressor protein (e.g. monomer or dimer);
L₂ is a peptide linker, such as a flexible peptide linker, such as a glycine/serine linker; and
X₃ comprises at least one nuclear localization signal.

8. The polynucleotide of any one of embodiments 1-7, wherein the CRO-NLS protein comprises a polypeptide having the formula of: X₁-L₁-X₂-L₂-X₃, wherein:
X₁ is an affinity binding domain;
X₂ is a CRO repressor protein;
X₃ comprises at least one nuclear localization signal or a synthetic bipartite nuclear localization signal; and
L₁ and L₂ are both, independently, flexible linker moieties, wherein L₁ and L₂ can be the same or different.

9. The polynucleotide molecule of any one of embodiments 1-6, wherein the CRO-NLS protein comprises a polypeptide having the formula of: X₂-L₂-X₃-L₃-X₄, wherein:
X₂ comprises the CRO protein,
X₃ comprises at least one nuclear localization signal or a synthetic bipartite nuclear localization signal,
X₄ comprises the C-terminus of bacteriophage repressor lambda, and
L₂ and L₃ are each, independently, flexible linker moieties, wherein L₂ and L₃ are the same or different.

10. The polynucleotide molecule of any one of embodiments 1-6, wherein the CRO-NLS protein comprises a polypeptide having the formula of: X₁-L₁-X₂-L₂-X₃-L₃-X₄, wherein:
X₁ comprises an affinity binding domain,
X₂ comprises the CRO protein,
X₃ comprises at least one nuclear localization signal or a synthetic bipartite nuclear localization signal,
X₄ comprises the C-terminus of bacteriophage repressor lambda, and
L₁, L₂, and L₃ are each, independently, flexible linker moieties, wherein L₁, L₂, and L₃ are the same or different.

11. The polynucleotide of any one of embodiments 1-6, wherein the CRO-NLS protein comprises a polypeptide having the formula of: X₂-L₂-X₅-L₃-X₃, wherein:
X₂ comprises a first CRO protein,
X₅ comprises a second CRO protein,
X₃ comprises at least one nuclear localization signal, and
L₂, and L₃ are all, independently, flexible linker moieties, wherein L₂ and L₃ can be the same or different, and wherein L₂ may optionally be a non-cleavable linker.

12. The polynucleotide of any one of embodiments 1-6, wherein the CRO-NLS protein comprises a polypeptide having the formula of: X₁-L₁-X₂-L₂-X₅-L₃-X₃, wherein:
X₁ comprises an affinity binding domain,
X₂ comprises a first CRO protein,
X₅ comprises a second CRO protein,
X₃ comprises at least one nuclear localization signal, and
L₁, L₂, and L₃ are all, independently, flexible linker moieties, wherein L₁, L₂ and L₃ can be the same or different, and wherein L₂ may optionally be a non-cleavable linker.

13. The polynucleotide of any one of embodiments 1-12, wherein the CRO protein comprises the amino acid sequence of:

(SEQ ID NO: 6)
MEQRITLKDYAMRFGQTKTAKDLGVYQSAINKAIHAGRKIFLTIN
ADGSVYAEEVKPFPSNKKTTA

14. The polynucleotide of any one of embodiments 1-13, wherein the NLS comprises the amino acid sequence of PAAKRVKLD (SEQ ID NO: 3); PKKKRKV (SEQ ID NO: 4); or PAAKRVKLDATESQDTGPPKKKRKV (SEQ ID NO: 5), or any combination thereof.

15. The polynucleotide of any one of embodiments 1-13, wherein the NLS comprises the synthetic bipartite nuclear localization signal with the amino acid sequence of

(SEQ ID NO: 5)
PAAKRVKLDATESQDTGPPKKKRKV.

16. The polynucleotide of any one of embodiments 7-12 wherein L₁, L₂ and L₃, as applicable, each, independently, comprises the amino acid sequence of (GGGGS)ₙ(SEQ ID NO: 22), (GGGSS)ₙ(SEQ ID NO: 23), (GSGSG)ₙ(SEQ ID NO: 24), (GSSG)ₙ(SEQ ID NO: 25), GPGSKLKSGFGGPGSRFRSGPG (SEQ ID NO: 1), or AAGGTGGGSGGGTGGS (SEQ ID NO: 2), or any combination thereof, wherein each n is, independently, 1-5.

17. The polynucleotide of any one of embodiments 7-12, wherein L₁, L₂ and L₃, as applicable, each, independently, comprises the amino acid sequence of MXGXG, IXGXG, LXGXG, MXGGX, IXGGX, LXGGX, wherein each X is any amino acid.

18. The polynucleotide of any one of embodiments 1-15, wherein the linker encoded by the polynucleotide is a peptide linker, including, but not limited to, GPGPG (SEQ ID NO: 32).

19. The polynucleotide of any one of embodiments 7-12, wherein L₂ comprises the amino acid sequence of: GPGSKLKSGFGGPGSRFRSGPG (SEQ ID NO: 1) or

(SEQ ID NO: 2)
AAGGTGGGSGGGTGGS.

20 The polynucleotide of embodiments 9 or 10, wherein, as applicable, the C-terminus of bacteriophage repressor lambda comprises the amino acid sequence of:

(SEQ ID NO: 7)
LRSEYEYPVFSHVQAGMFSPELRTFTKGDAERWVSTTKKASDSAF
WLEVEGNSMTAPTGSKPSFPDGMLILVDPEQAVEPGDFCIARLGG
DEFTFKKLIRDSGQVFLQPLNPQYPMIPCNESCSVVGKVIASQWP
EETFG.

21. The polynucleotide of any one of embodiments 1-20, wherein, as applicable, the affinity binding domain is a heterologous tag.

22. The polynucleotide of embodiment 21, wherein the heterologous tag is a Flag tag, CBP tag, HA tag, HBH tag, Myc tag, histidine tag, S-tag, TAP, V5, or any combination thereof.

23. The polynucleotide of any one of embodiments 1-22, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 8, or comprises SEQ ID NO: 8.

24. The polynucleotide molecule of any one of embodiments 1-23, wherein the nucleotide sequence encoding for the CRO-NLS protein comprises a nucleic acid sequence having at least 85%, 86%, 87%, 88%, 89%, 90%, 91%, 92%, 93%, 94%, 95%, 96%, 97%, 98%, or 99% identity to SEQ ID NO: 9, or comprises the sequence of SEQ ID NO: 9.

25. The polynucleotide molecule of any one of embodiments 1-22, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 10, or comprises the sequence of SEQ ID NO: 10.

26 The polynucleotide molecule of any one of embodiments 1-22 or 25, wherein the sequence encoding for the CRO-NLS protein comprises a nucleic acid sequence having at least 85%, 86%, 87%, 88%, 89%, 90%, 91%, 92%, 93%, 94%, 95%, 96%, 97%, 98%, or 99% identity to SEQ ID NO: 11, or comprises the sequence of SEQ ID NO: 11.

27 The polynucleotide molecule of any one of embodiments 1-22, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 12, or comprises the sequence of SEQ ID NO: 12.

28. The polynucleotide molecule of any one of embodiments 1-22 or 27, wherein the sequence encoding for the CRO-NLS protein comprises a nucleic acid sequence having at least 85%, 86%, 87%, 88%, 89%, 90%, 91%, 92%, 93%, 94%, 95%, 96%, 97%, 98%, or 99% identity to SEQ ID NO: 13, or comprises the sequence of SEQ ID NO: 13.

29. The polynucleotide molecule of any one of embodiments 1-22, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 14, or comprises a sequence of SEQ ID NO: 14.

30. The polynucleotide molecule of any one of embodiments 1-22 or embodiment 29, wherein the sequence encoding for the CRO-NLS protein comprises a nucleic acid sequence having at least 85%, 86%, 87%, 88%, 89%, 90%, 91%, 92%, 93%, 94%, 95%, 96%, 97%, 98%, or 99% identity to SEQ ID NO: 15, or comprises the sequence of SEQ ID NO: 15.

31. The polynucleotide of any one of embodiments 1-30, wherein the at least one promoter region is upstream of the sequence encoding a CRO-NLS protein.

32. A polypeptide encoded by the polynucleotide of any one of embodiments 1-31.

33. A polypeptide, including an isolated polypeptide, comprising a CRO repressor protein operably connected to a nuclear localization signal (“CRO-NLS protein”).

34 The polypeptide of embodiment 33, the CRO repressor protein is linked to the NLS by a linker.

35 The polypeptide of embodiment 34, wherein the linker is a peptide linker, such as, but not limited to, a glycine/serine linker, including, but not limited to, (GGGGS)ₙ(SEQ ID NO: 22), (GGGSS)ₙ(SEQ ID NO: 23), (GSGSG)ₙ(SEQ ID NO: 24), (GSSG)ₙ(SEQ ID NO: 25), GPGSKLKSGFGGPGSRFRSGPG (SEQ ID NO: 1), or AAGGTGGGSGGGTGGS (SEQ ID NO: 2), or any combination thereof, wherein each n is, independently, 1-5.

36 The polypeptide of any one of embodiments 33-35, wherein the CRO-NLS comprises a monomer or dimer of a CRO repressor protein.

37 The polypeptide of any one of embodiments 33-36, wherein the CRO-NLS protein comprises a polypeptide having a formula of: X₂-L₂-X₃, wherein:
X₂ is a CRO repressor protein (e.g. monomer or dimer);
L₂ is a peptide linker, such as a flexible peptide linker, such as a glycine/serine linker;
and
X₃ comprises at least one nuclear localization signal.

38. The polypeptide of any one of embodiments 33-36, wherein the CRO-NLS protein comprises a polypeptide having the formula of: X₁-L₁-X₂-L₂-X₃, wherein:
X₁ is an affinity binding domain;
X₂ is a CRO repressor protein;
X₃ comprises at least one nuclear localization signal or a synthetic bipartite nuclear localization signal; and
L₁ and L₂ are both, independently, flexible linker moieties, wherein L₁ and L₂ can be the same or different.

39 The polypeptide of any one of embodiments 33-36, wherein the CRO-NLS protein comprises a polypeptide having the formula of: X₂-L₂-X₃-L₃-X₄, wherein:
X₂ comprises the CRO protein,
X₃ comprises at least one nuclear localization signal or a synthetic bipartite nuclear localization signal,
X₄ comprises the C-terminus of bacteriophage repressor lambda, and
L₂ and L₃ are each, independently, flexible linker moieties, wherein L₂ and L₃ are the same or different.

40. The polypeptide of any one of embodiments 33-36, wherein the CRO-NLS protein comprises a polypeptide having the formula of: X₁-L₁-X₂-L₂-X₃-L₃-X₄, wherein:
X₁ comprises an affinity binding domain,
X₂ comprises the CRO protein,
X₃ comprises at least one nuclear localization signal or a synthetic bipartite nuclear localization signal,
X₄ comprises the C-terminus of bacteriophage repressor lambda, and
L₁, L₂, and L₃ are each, independently, flexible linker moieties, wherein L₁, L₂, and L₃ are the same or different.

41. The polypeptide of any one of embodiments 33-36, wherein the CRO-NLS protein comprises a polypeptide having the formula of: X₂-L₂-X₅-L₃-X₃, wherein:
X₂ comprises a first CRO protein;
X₅ comprises a second CRO protein;
X₃ comprises at least one nuclear localization signal; and
L₂, and L₃ are all, independently, flexible linker moieties, wherein L₁, L₂, and L₃ may comprise the same or unique linker sequences, and wherein L₂ may optionally be a non-cleavable linker.

42 The polypeptide of any one of embodiments 33-36, wherein the CRO-NLS protein comprises a polypeptide having the formula of: X₁-L₁-X₂-L₂-X₅-L₃-X₃, wherein:
X₁ comprises an affinity binding domain,
X₂ comprises a first CRO protein,
X₅ comprises a second CRO protein,
X₃ comprises at least one nuclear localization signal, and
L₁, L₂, and L₃ are all, independently, flexible linker moieties, wherein L₁, L₂, and L₃ may comprise the same or unique linker sequences, and wherein L₂ may optionally be a non-cleavable linker.

43. The polypeptide of embodiments 41 or 42, wherein the first CRO protein and the second CRO protein are the same or different.

44. The polypeptide of any one of embodiments 33-43, wherein the CRO protein comprises the amino acid sequence of:

(SEQ ID NO: 6)
MEQRITLKDYAMRFGQTKTAKDLGVYQSAINKAIHAGRKIFLTIN
ADGSVYAEEVKPFPSNKKTTA.

45. The polypeptide of any one of embodiments 33-44, wherein the NLS comprises the amino acid sequence of PAAKRVKLD (SEQ ID NO: 3); PKKKRKV (SEQ ID NO: 4); or PAAKRVKLDATESQDTGPPKKKRKV (SEQ ID NO: 5), or any combination thereof.

46. The polypeptide of any one of embodiments 33-45, wherein L₁, L₂, and L₃, as applicable, each, independently, comprises the amino acid sequence of (GGGGS)ₙ(SEQ ID NO: 22), (GGGSS)ₙ(SEQ ID NO: 23), (GSGSG)ₙ(SEQ ID NO: 24), (GSSG)ₙ(SEQ ID NO: 25), GPGSKLKSGFGGPGSRFRSGPG (SEQ ID NO: 1), or AAGGTGGGSGGGTGGS (SEQ ID NO: 2), or any combination thereof, wherein each n is, independently, 1-5.

47. The polypeptide of any one of embodiments 33-45, wherein L₁, L₂ and L₃, as applicable, each, independently, comprises the amino acid sequence of MXGXG, IXGXG, LXGXG, MXGGX, IXGGX, LXGGX, wherein each X is any amino acid.

48. The polypeptide of any one of embodiments 33-45, wherein L₁, L₂ and L₃, as applicable, each, independently, comprises an amino acid sequence of, including, but not limited to, GPGPG (SEQ ID NO: 32).

49 The polypeptide of embodiments 41 or 42, wherein L₂ comprises the amino acid sequence of: GPGSKLKSGFGGPGSRFRSGPG (SEQ ID NO: 1) or AAGGTGGGSGGGTGGS (SEQ ID NO: 2).

50. The polypeptide of embodiments 39 or 40, wherein the C-terminus of bacteriophage repressor lambda comprises the amino acid sequence of:

(SEQ ID NO: 7)
LRSEYEYPVFSHVQAGMFSPELRTFTKGDAERWVSTTKKASDSAF
WLEVEGNSMTAPTGSKPSFPDGMLILVDPEQAVEPGDFCIARLGG
DEFTFKKLIRDSGQVFLQPLNPQYPMIPCNESCSVVGKVIASQWP
EETFG.

51. The polypeptide of any one of embodiments 33-50, wherein, as applicable, the affinity binding domain is a heterologous tag.

52. The polypeptide of embodiment 51, wherein the heterologous tag is a Flag tag, CBP tag, HA tag, HBH tag, Myc tag, histidine tag, S-tag, TAP, V5, or any combination thereof.

53 The polypeptide of any one of embodiments 33-52, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 8, or comprises SEQ ID NO: 8.

54. The polypeptide of any one of embodiments 33-52, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 10, or comprises the sequence of SEQ ID NO: 10.

55. The polypeptide of any one of embodiments 33-52, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 12, or comprises the sequence of SEQ ID NO: 12.

56 The polynucleotide molecule of any one of embodiments 33-53, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 14, or comprises a sequence of SEQ ID NO: 14.

57. A cell comprising the polynucleotide of any one of embodiments 1-32 and a cargo polynucleotide comprising a nucleic acid sequence encoding for a molecule of interest, a promoter region operably connected to the molecule of interest, and at least a first CRO repressor binding site.

58. A cell comprising the polypeptide of any one of embodiments 33-56 and a cargo polynucleotide comprising a nucleic acid sequence encoding for a molecule of interest, a promoter region operably connected to the molecule of interest, and at least a first CRO repressor binding site.

59. The cell of embodiments 57 or 58, wherein the CRO repressor binding site is operably connected to the promoter region of the cargo polynucleotide, wherein in the presence of a CRO protein, the CRO protein inhibits the expression (e.g., transcription or translation) of the molecule of interest by binding to the CRO repressor binding site.

60. The cell of any one of embodiments 57-59, wherein the cargo polynucleotide comprises a nucleic acid sequence encoding for the molecule of interest, the promoter region operably connected to the molecule of interest, and the least a first CRO repressor binding site.

61. The cell of any one of embodiments 57-60, wherein the cargo polynucleotide comprises a 5′ adenoviral ITR and a 3′ adenoviral ITR, wherein the 5′ ITR and 3′ ITR flank the nucleic acid sequence encoding for the molecule of interest, the promoter region operably connected to the molecule of interest, and the least a first CRO repressor binding site.

62. The cell of any one of embodiments 57-61, wherein the promoter region of the cargo polynucleotide is located upstream of the sequence encoding the molecule of interest

63. The cell of any one of embodiments 57-62, wherein the at least a first CRO repressor binding site is located upstream of the sequence encoding for the molecule of interest.

64. The cell of any one of embodiments 57-63, wherein the at least a first CRO repressor binding site is located upstream of the promoter region of the cargo polynucleotide.

65. The cell of any one of embodiments 57-63, wherein the at least a first CRO repressor binding site is located between the promoter region and the sequence encoding for the molecule of interest of the cargo polynucleotide.

66. The cell of any one of embodiments 57-63, wherein the at least a first CRO repressor binding site is located within the promoter region of the cargo polynucleotide.

67. The cell of any one of embodiments 57-66, wherein the at least a first CRO repressor binding site is selected from the group consisting of rightward operators of a bacteriophage and leftward operators of a bacteriophage.

68. The cell of any one of embodiments 57-67 wherein the at least a first CRO repressor binding site is selected from the group consisting of rightward operators of bacteriophage λ and leftward operators of bacteriophage 2.

69. The cell of any one of embodiments 57-68, wherein the at least a first CRO repressor binding site is selected from the group consisting of a bacteriophage A leftward operator 1 (O_{L1}), a bacteriophage A leftward operator 2 (O_{L2}), a bacteriophage λ leftward operator 3 (O_{L3}), a bacteriophage λ rightward operator 1 (O_{R1}), a bacteriophage λ rightward operator 2 (O_{R2}), and a bacteriophage λ rightward operator 3 (O_{R3}).

70. The cell of any one of embodiments 57-69, wherein the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 16, or comprises a sequence of SEQ ID NO: 16.

71. The cell of any one of embodiments 57-69, wherein the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 17, or comprises a sequence of SEQ ID NO: 17.

72. The cell of any one of embodiments 57-69, wherein the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 18, or comprises a sequence of SEQ ID NO: 18.

73. The cell of any one of embodiments 57-69, wherein the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19, or comprises a sequence of SEQ ID NO: 19.

74. The cell of any one of embodiments 57-69, wherein the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 20, or comprises a sequence of SEQ ID NO: 20.

75. The cell of any one of embodiments 57-69, wherein the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 21, or comprises a sequence of SEQ ID NO: 21.

76. The cell of any one of embodiments, 57-75, wherein the cargo polynucleotide comprises a second CRO repressor binding site.

77 The cell of embodiment 76, wherein the at least first and the second CRO repressor binding sites are upstream of the sequence encoding for the molecule of interest.

78 The cell of embodiment 76, wherein the at least first and the second CRO repressor binding sites are upstream of the promoter region.

79. The cell of embodiment 76, wherein the least first and the second CRO repressor binding sites are between the promoter region and the sequence encoding for the molecule of interest.

80. The cell of embodiment 76, wherein the at least first CRO repressor binding site is located within the promoter region and the second CRO repressor binding site is located between the promoter region and the sequence encoding for the molecule of interest.

81. The cell of embodiment 76, wherein the at least first CRO repressor binding site is located upstream of the promoter region and the second CRO repressor binding site is located within the promoter region.

82. The cell of any one of embodiments 76-81, wherein the second CRO repressor binding site is selected from the group consisting of rightward operators of a bacteriophage and leftward operators of a bacteriophage.

83. The cell of any one of embodiments 76-82, wherein the second CRO repressor binding site is selected from the group consisting of rightward operators of bacteriophage λ and leftward operators of bacteriophage 2.

84. The cell of any one of embodiments 76-83, wherein the second CRO repressor binding site is selected from the group consisting of a bacteriophage λ leftward operator 1 (O_{L1}), a bacteriophage A leftward operator 2 (O_{L2}), a bacteriophage A leftward operator 3 (O_{L3}), a bacteriophage λ rightward operator 1 (O_{R1}), a bacteriophage λ rightward operator 2 (O_{R2}), and a bacteriophage λ rightward operator 3 (O_{R3}).

85. The cell of any one of embodiments 76-84, wherein the second CRO repressor binding site comprises:
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 16, or comprises a sequence of SEQ ID NO: 16;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 17, or comprises a sequence of SEQ ID NO: 17;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 18, or comprises a sequence of SEQ ID NO: 18;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19 or comprises a sequence of SEQ ID NO: 19;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 20 or a comprises a sequence of SEQ ID NO: 20; or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 21 or a comprises a sequence of SEQ ID NO: 21.

86 The cell of any one of embodiments 57-85, wherein the sequence encoding the molecule of interest encodes for one or more of: a viral protein, a shRNA, therapeutic molecule, a tumor antigen, a protein, a nucleic acid molecule, or a combination thereof.

87. The cell of embodiment 86, wherein the therapeutic molecule is a cytokine, such as IL-2, IL-12, IL-15, and the like.

88. A virus comprising a cargo polynucleotide comprising a nucleic acid sequence encoding for a molecule of interest, a promoter region operably connected to the molecule of interest, and at least a first CRO repressor binding site.

89 The virus of embodiment 88, wherein the cargo polynucleotide comprises a 5′ adenoviral ITR and a 3′ adenoviral ITR, wherein the 5′ ITR and 3′ ITR flank the nucleic acid sequence encoding for the molecule of interest, the promoter region operably connected to the molecule of interest, and the least a first CRO repressor binding site.

90 The virus of embodiments 88 and 89, wherein the promoter region of the cargo polynucleotide is located upstream of the sequence encoding the molecule of interest.

91. The virus of any one of embodiments 88-90, wherein:
the at least a first CRO repressor binding site is located upstream of the sequence encoding for the molecule of interest;
the at least a first CRO repressor binding site is located upstream of the promoter region of the cargo polynucleotide;
the at least a first CRO repressor binding site is located between the promoter region and the sequence encoding for the molecule of interest of the cargo polynucleotide; or
the at least a first CRO repressor binding site is located within the promoter region of the cargo polynucleotide.

92. The virus of any one of embodiments 88-91, wherein the at least a first CRO repressor binding site is selected from the group consisting of rightward operators of a bacteriophage and leftward operators of a bacteriophage.

93. The virus of any one of embodiments 88-92, wherein the at least a first CRO repressor binding site is selected from the group consisting of rightward operators of bacteriophage λ and leftward operators of bacteriophage 2.

94 The virus of any one of embodiments 88-93, wherein the at least a first CRO repressor binding site is selected from the group consisting of a bacteriophage A leftward operator 1 (O_{L1}), a bacteriophage λ leftward operator 2 (O_{L2}), a bacteriophage A leftward operator 3 (O_{L3}), a bacteriophage λ rightward operator 1 (O_{R1}), a bacteriophage λ rightward operator 2 (O_{R2}), and a bacteriophage λ rightward operator 3 (O_{R3}).

95 The virus of any one of embodiments 88-94, wherein the at least a first CRO repressor binding site comprises:
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 16, or comprises a sequence of SEQ ID NO: 16;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 17, or comprises a sequence of SEQ ID NO: 17;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 18, or comprises a sequence of SEQ ID NO: 18;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19, or comprises a sequence of SEQ ID NO: 19;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 20, or comprises a sequence of SEQ ID NO: 20;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 21, or comprises a sequence of SEQ ID NO: 21.

96. The virus of any one of embodiments, 88-95, wherein the cargo polynucleotide comprises a second CRO repressor binding site.

97. The virus of embodiment 96, wherein:
the at least first and the second CRO repressor binding sites are upstream of the sequence encoding for the molecule of interest;
the at least first and the second CRO repressor binding sites are upstream of the promoter region;
the least first and the second CRO repressor binding sites are between the promoter region and the sequence encoding for the molecule of interest;
the at least first CRO repressor binding site is located within the promoter region and the second CRO repressor binding site is located between the promoter region and the sequence encoding for the molecule of interest; or the at least first CRO repressor binding site is located upstream of the promoter region and the second CRO repressor binding site is located within the promoter region.

98 The virus of any one of embodiments 96-97, wherein the second CRO repressor binding site is selected from the group consisting of rightward operators of a bacteriophage and leftward operators of a bacteriophage.

99. The virus of any one of embodiments 96-98, wherein the second CRO repressor binding site is selected from the group consisting of rightward operators of bacteriophage λ and leftward operators of bacteriophage 2.

100. The virus of any one of embodiments 96-99, wherein the second CRO repressor binding site is selected from the group consisting of a bacteriophage λ leftward operator 1 (O_{L1}), a bacteriophage A leftward operator 2 (O_{L2}), a bacteriophage A leftward operator 3 (O_{L3}), a bacteriophage λ rightward operator 1 (O_{R1}), a bacteriophage λ rightward operator 2 (O_{R2}), and a bacteriophage λ rightward operator 3 (O_{R3}).

101. The virus of any one of embodiments 96-100, wherein the second CRO repressor binding site comprises:
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 16, or comprises a sequence of SEQ ID NO: 16;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 17, or comprises a sequence of SEQ ID NO: 17;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 18, or comprises a sequence of SEQ ID NO: 18;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19 or comprises a sequence of SEQ ID NO: 19;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 20 or a comprises a sequence of SEQ ID NO: 20;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 21 or a comprises a sequence of SEQ ID NO: 21.

102. The virus of any one of embodiments 88-101, wherein the sequence encoding the molecule of interest encodes for one or more of: a viral protein, a shRNA, therapeutic molecule, a tumor antigen, a protein, a nucleic acid molecule, or a combination thereof.

103. The virus of embodiment 102, wherein the therapeutic molecule is a cytokine, such as IL-2, IL-12, IL-15, antigen, tumor antigen, protein, viral antigen, and the like.

104. The virus of embodiment 102, wherein the therapeutic molecule is any recombinant protein or RNA, including, but not limited, to a synthetic polytope.

105. A method of making a virus comprising the cargo polynucleotide, the method comprising culturing the cell of any one of embodiments 57-87 under conditions to produce the virus.

106. The method of embodiment 105, wherein the virus that is produced is an adenovirus.

107. The method of embodiments 105 or 106, wherein the virus is replication incompetent or replication competent.

108. A virus prepared according to a method of any one of embodiments 105-107.

109. A plasmid comprising the polynucleotide of any one of embodiments 1-31.

110. A cell comprising the polynucleotide of any one of embodiments 1-31 or the plasmid of embodiment 109.

111. A method of controlling expression of a molecule of interest, comprising:
contacting a host cell comprising a polynucleotide of any one of embodiments 1-31 or a polypeptide of any one of embodiments 32-56 with a cargo polynucleotide comprising a nucleic acid sequence encoding for a molecule of interest, a promoter region operably connected to the molecule of interest, and at least a first CRO repressor binding site,
wherein:
the expression of the molecule of interest is controlled by the binding of the polypeptide encoded for by the polynucleotide of any one of embodiments 1-31 or the polypeptide of any one of embodiments 32-56 to the CRO repressor binding site.

112. The method of embodiment 111, wherein the molecule of interest is one or more of: a viral protein, a shRNA, therapeutic molecule, a tumor antigen, a protein, a nucleic acid molecule, or a combination thereof.

113. A method of delivering a molecule of interest to a subject, the method comprising administering the virus of any one of embodiments 88-104 to the subject.

114. A method of inducing an immune response in a subject against a molecule of interest, the method comprising administering the virus of any one of embodiments 88-104 to the subject, wherein the molecule of interest is a viral protein or a tumor antigen.

115. A method of treating a disease, the method comprising administering the virus of any one of embodiments 88-104 to a subject to treat the disease.

116. The method of embodiment 115, wherein the disease is an infectious disease or a cancer.

117. A polypeptide comprising a nuclear localization signal (NLS) linked or fused to a heterologous molecule, wherein the NLS is a polypeptide having the formula of NLS₁-Xₙ-NLS₂, wherein NLS₁ and NLS₂ can comprise the same or different NLS sequences, Xₙ is a peptide linker, such as described herein.

118. The polypeptide of embodiment 117, wherein NLS₁ and NLS₂ are each, independently, selected from the group consisting of SEQ ID NO: 3, SEQ ID NO: 4, or a combination thereof.

119. The polypeptide of embodiments 117 or 118, wherein NLS₁ is SEQ ID NO: 3 and NLS₂ is SEQ ID NO: 4.

120. The polypeptide of any one of embodiments 117-119 wherein n is 9.

121. The polypeptide of any one of embodiments 117-120, wherein Xₙ comprises the amino acid sequence of ATESQDTGP (SEQ ID NO: 33).

122. The polypeptide of any one of embodiments 117-121, wherein the NLS comprises an amino acid sequence 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 5, or comprises a sequence of SEQ ID NO: 5.

123. The polypeptide of any one of embodiments 117-122, wherein the NLS comprises SEQ ID NO: 5.

124. The polypeptide of any one of embodiments 117-123, wherein the heterologous molecule is a polypeptide.

125. The polypeptide of any one of embodiments 117-123, wherein the heterologous molecule comprises a nucleic acid molecule.

126. The polypeptide of any one of embodiments 117-125, wherein the NLS and the heterologous molecule are joined or connected with a linker.

127. The polypeptide of embodiment 126, wherein the linker is a peptide linker, such as a peptide linker comprising the amino acid sequence of (GGGGS)ₙ(SEQ ID NO: 22), (GGGSS)ₙ (SEQ ID NO: 23), (GSGSG)ₙ(SEQ ID NO: 24), (GSSG)ₙ(SEQ ID NO: 25), GPGSKLKSGFGGPGSRFRSGPG (SEQ ID NO: 1), AAGGTGGGSGGGTGGS (SEQ ID NO: 2), MXGXG, IXGXG, LXGXG, MXGGX, IXGGX, LXGGX, GPGPG (SEQ ID NO: 32), or any combination thereof, wherein each n is, independently, 1-5 and wherein X is any amino acid.

128. The polypeptide of any one of embodiments 117-127, wherein the polypeptide further comprises a second heterologous molecule.

129. A polynucleotide molecule encoding the polypeptide of any one of embodiments 117-128.

130. The polynucleotide of embodiment 129, wherein the polynucleotide encoding the polypeptide is operably linked to a promoter.

131. A plasmid, cell, or virus comprising the polynucleotide molecule of embodiments 129 or 130.

132. A cell comprising the polypeptide of any one of embodiments 117-128.

133. A method of transporting a heterologous molecule of interest to the nucleus of a cell, the method comprising contacting the cell with a polypeptide of any one of embodiments 117-128

134. A method for transporting a heterologous molecule of interest to the nucleus of a cell, the method comprising contacting the cell with a polynucleotide of embodiments 129 or 130 or with a plasmid comprising the same under conditions sufficient to express the molecule in the cell.

135. A method for transporting a heterologous molecule of interest to the nucleus of a cell, the method comprising contacting the cell with a vector comprising the polynucleotide of embodiments 129 or 130.

### Examples

The following examples are illustrative, but not limiting, of the compounds, compositions, and methods described herein. Other suitable modifications and adaptations known to those skilled in the art are within the scope of the following examples.

### Example 1: NLS Targets CRO to the Nucleus in Mammalian Cells

A vector construct comprising a polynucleotide comprising a CRO-NLS construct as described herein will be delivered to a mammalian cell and allowed to incubate for a set time. Proper expression of the CRO-NLS construct in the nucleus will be verified via affinity antibody assays, such as western blot on nuclear and cytoplasmic fractions or confocal microscopy. As a control, CRO constructs in the absence of NLS will also be used.

### Example 2: Controlling Expression of a Molecule of Interest

A vector construct will be provided, where said vector construct comprises at least a promoter region, at least a first CRO repressor binding site, and a multiple cloning site. Both the promoter region and the at least a first CRO repressor binding site are located upstream of the multiple cloning site. The location of the promoter region with respect to the at least a first CRO repressor binding site will be varied as described in embodiments herein. Several vector constructs will be tested to determine the effect of positional variability of the at least a first CRO repressor binding site on the expression of a molecule of interest that will be inserted into the multiple cloning site. For these experiments, the molecule of interest will be a molecule readily detected by various standard laboratory techniques (e.g. GFP and the like or another affinity tagged protein). As a control, vector constructs that do not contain the at least a first CRO repressor binding site will also be utilized.

A polynucleotide encoding a molecule of interest will be introduced into the CRO repressor binding site and control expression vectors via standard cloning. The various CRO repressor binding site and control vector constructs will be delivered to mammalian cells in culture and allowed to incubate for a set time. The mammalian cells will, either simultaneously or previously, also be contacted with a vector construct encoding a CRO-NLS construct. Alternatively, the mammalian cell will already contain the CRO-NLS construct within its genome.

The ability of the CRO-NLS construct to control the expression of GFP or another affinity tagged protein will be assessed via standard laboratory techniques, i.e. western blotting or confocal microscopy. The relative effect of CRO-NLS on the expression of GFP and the like or another affinity tagged protein will be determined via comparison to the control vectors that do not contain the at least a first CRO repressor binding site.

### Example 3: Controlling Expression of a Toxic Molecule of Interest

Experiments will be carried out in a similar manner to those outlined in example 2, except that the molecule of interest will be selected such that expression of the molecule of interest is toxic to the cell. Experimental readouts will be standard cellular toxicity assay, for example MTT assay or other fluorometric cell viability readouts.

### Example 4: Production of Viruses Comprising a Molecule of Interest

Experiments will be carried out in a similar manner to those outlined in example 2, however the vector construct comprising the at least a first CRO repressor binding site will be a viral vector construct and the nucleic acid sequence encoding for the molecule of interest, the promoter region, and the at least a first CRO repressor binding site will be flanked by a 5′ adenoviral ITR and a 3′ adenoviral ITR. Additionally, the cell chosen will further comprise the components necessary to construct a virus.

The molecule of interest will again be GFP and the like or another affinity tagged protein, and the ability of the at least a first CRO repressor binding site to prevent the leaky expression of GFP and the like or another affinity tagged protein during the production of the viruses will be assessed. Control viral vectors not containing the at least a first CRO repressor binding site will also be utilized. The ability to prevent leaky expression of GFP and the like or another affinity tagged protein will be assessed via comparison to viral preparations using control constructs.

### Example 5: Production of Viruses Comprising a Toxic Molecule of Interest

Experiments will be carried out in a similar manner to those outlined in example 4, except the molecule of interest will be selected such that the expression of the molecule of interest is toxic to the cell. Experimental readouts will be standard cellular toxicity assay, for example MTT assay or other fluorometric cell viability readouts. Additionally, final viral titer will be assessed, as prevention of the toxicity of a molecule of interest will allow the cells to produce more viral particles over a longer period of time.

### Example 6: Viruses Produced Under CRO Control Express the Molecule of Interest in the Absence of CRO

Viruses will be prepared as outlined in example 4 or example 5. The viruses will then be used to deliver the GFP and the like or another affinity tagged protein to a separate cell population, wherein the cell population does not comprise the CRO-NLS machinery. As a control, the viruses will also be delivered to cells that do contain the CRO-NLS machinery. Expression of GFP and the like or another affinity tagged protein will be compared between the two cell populations. Although expression of GFP and the like or another affinity tagged protein during production of the viruses is expected to be controlled, expression of GFP and the like or another affinity tagged protein is expected to be restored when the virus is delivered to a cell that does not comprise the CRO-NLS machinery.

### Example 7: Delivery of a Molecule of Interest to a Target Cell in a Subject

Viruses will be prepared as outlined in examples 4-6. The virus will then be used to deliver GFP and the like or another affinity tagged protein to a subject (e.g., mouse) and the expression of GFP and the like or another affinity tagged protein in the target cell population will be assessed.

### Example 8: Inducing an Immune Response in a Subject

Viruses will be prepared as outlined in examples 4-7. The molecule of interest will be a cytokine, such as IL-2, IL-12, IL-15, antigen, tumor antigen, protein, viral antigen, and the like. The virus will be delivered to the subject (e.g. mouse).

Expression of the molecule of interest in the target cells will be confirmed via standard laboratory techniques. The immune response elicited via delivery of the virus will also be assessed. As a control, a virus delivering a molecule that does not elicit a substantial immune response (e.g. GFP) will also be prepared and administered to the subject.

### Example 9: Treatment of Cancer in a Subject

Viruses will be prepared as outlined in example 4-8. The molecule of interest will be selected such that expression of the molecule of interest in the target cell is expected to cause cytotoxicity. The target cell is a cancer cell. The virus will be delivered to the subject, wherein the virus will transduce the target cancer cell with the cytotoxic molecule of interest, thereby killing the cancer cell and treating the cancer.

### Example 10: NLS Sequence Directs Cargo to the Nucleus of a Cell

A fusion protein comprising the NLS of SEQ ID NO: 5 is expressed in a cell and found to traffic the fusion molecule to the nucleus.

### Example 11: CRO-Based Inhibition of a CRO-Regulated Reporter Construct

To test the ability of various CRO-NLS proteins described herein to regulate the expression of molecules of interest, the following constructs were transfected into mammalian HEK cells along with a mammalian expression construct containing a minimal CMV promoter and O_{R} sequences from bacteriophage lambda together with a reporter protein (GFP):
a monomeric CRO-NLS protein (Cro);
a dimeric CRO-NLS protein (DiCro);
a dimeric CRO-NLS protein with cleavable linker (DiCroCl);
and a CRO-NLS-bacteriophage repressor lambda protein (CroLambdaR).
   The GFP expression of these systems was compared to a reporter construct using a constitutive promoter linked to GFP. The results listing the percentage of GFP-positive cells, normalized to the constitutively active GFP control are shown in Table 1. The results show that each CRO-NLS system is able to inhibit GFP production compared to the constitutive control, with different CRO-NLS proteins resulting in different levels of GFP inhibition.

TABLE 1

| | GFP control | Cro | DiCro | DiCroCl | CroLambdaR |
|---|---|---|---|---|---|
| Clone 1 | 99.8 | 80.2 | 65.1 | 32.0 | 17.8 |
| Clone 2 | 96.4 | 85.8 | 70.4 | 58.0 | 53.8 |
| Average | 98.1 | 83.0 | 67.8 | 45.0 | 35.8 |

What is claimed is:

1. A polynucleotide comprising at least one promoter region operably connected to a nucleotide sequence encoding for a CRO repressor protein operably connected to a nuclear localization signal (“CRO-NLS protein”).

2. The polynucleotide of claim 1, wherein the nucleotide sequence encoding for the CRO-NLS protein encodes a CRO repressor protein linked to the NLS by a linker.

3. The polynucleotide of claim 2, wherein the linker encoded by the polynucleotide is a peptide linker, such as a glycine/serine linker, including, but not limited to, (GGGGS)ₙ(SEQ ID NO: 22), (GGGSS)ₙ(SEQ ID NO: 23), (GSGSG)ₙ(SEQ ID NO: 24), (GSSG)ₙ(SEQ ID NO: 25), GPGSKLKSGFGGPGSRFRSGPG (SEQ ID NO: 1), or AAGGTGGGSGGGTGGS (SEQ ID NO: 2), or any combination thereof, wherein each n is, independently, 1-5.

4. The polynucleotide of claim 2, wherein the linker encoded by the polynucleotide is a peptide linker, including, but not limited to, MXGXG, IXGXG, LXGXG, MXGGX, IXGGX, LXGGX, wherein each X is any amino acid.

5. The polynucleotide of claim 2, wherein the linker encoded by the polynucleotide is a peptide linker, including, but not limited to, GPGPG (SEQ ID NO: 32).

6. The polynucleotide of any one of claims 1-5, wherein CRO-NLS protein encoded by the nucleotide sequence comprises a monomer or dimer of a CRO repressor protein.

7. The polynucleotide of any one of claims 1-6, wherein the CRO-NLS protein comprises a polypeptide having a formula of: X₂-L₂-X₃, wherein:
X₂ is a CRO repressor protein (e.g. monomer or dimer);
L₂ is a peptide linker, such as a flexible peptide linker, such as a glycine/serine linker; and
X₃ comprises at least one nuclear localization signal.

8. The polynucleotide of any one of claims 1-7, wherein the CRO-NLS protein comprises a polypeptide having the formula of: X₁-L₁-X₂-L₂-X₃, wherein:
X₁ is an affinity binding domain;
X₂ is a CRO repressor protein;
X₃ comprises at least one nuclear localization signal or a synthetic bipartite nuclear localization signal; and
L₁ and L₂ are both, independently, flexible linker moieties, wherein L₁ and L₂ can be the same or different.

9. The polynucleotide molecule of any one of claims 1-6, wherein the CRO-NLS protein comprises a polypeptide having the formula of: X₂-L₂-X₃-L₃-X₄, wherein:
X₂ comprises the CRO protein,
X₃ comprises at least one nuclear localization signal or a synthetic bipartite nuclear localization signal,
X₄ comprises the C-terminus of bacteriophage repressor lambda, and
L₂ and L₃ are each, independently, flexible linker moieties, wherein L₂ and L₃ are the same or different.

10. The polynucleotide molecule of any one of claims 1-6, wherein the CRO-NLS protein comprises a polypeptide having the formula of: X₁-L₁-X₂-L₂-X₃-L₃-X₄, wherein:
X₁ comprises an affinity binding domain,
X₂ comprises the CRO protein,
X₃ comprises at least one nuclear localization signal or a synthetic bipartite nuclear localization signal,
X₄ comprises the C-terminus of bacteriophage repressor lambda, and
L₁, L₂, and L₃ are each, independently, flexible linker moieties, wherein L₁, L₂, and L₃ are the same or different.

11. The polynucleotide of any one of claims 1-6, wherein the CRO-NLS protein comprises a polypeptide having the formula of: X₂-L₂-X₅-L₃-X₃, wherein:
X₂ comprises a first CRO protein,
X₅ comprises a second CRO protein,
X₃ comprises at least one nuclear localization signal, and
L₂, and L₃ are all, independently, flexible linker moieties, wherein L₂ and L₃ can be the same or different, and wherein L₂ may optionally be a non-cleavable linker.

12. The polynucleotide of any one of claims 1-6, wherein the CRO-NLS protein comprises a polypeptide having the formula of: X₁-L₁-X₂-L₂-X₅-L₃-X₃, wherein:
X₁ comprises an affinity binding domain,
X₂ comprises a first CRO protein,
X₅ comprises a second CRO protein,
X₃ comprises at least one nuclear localization signal, and
L₁, L₂, and L₃ are all, independently, flexible linker moieties, wherein L₁, L₂ and L₃ can be the same or different, and wherein L₂ may optionally be a non-cleavable linker.

13. The polynucleotide of any one of claims 1-12, wherein the CRO protein comprises the amino acid sequence of:
(SEQ ID NO: 6)
MEQRITLKDYAMRFGQTKTAKDLGVYQSAINKAIHAGRKIFLTIN
ADGSVYAEEVKPFPSNKKTTA.

14. The polynucleotide of any one of claims 1-13, wherein the NLS comprises the amino acid sequence of PAAKRVKLD (SEQ ID NO: 3); PKKKRKV (SEQ ID NO: 4); or
PAAKRVKLDATESQDTGPPKKKRKV (SEQ ID NO: 5), or any combination thereof.

15. The polynucleotide of any one of claims 1-13, wherein the NLS comprises the synthetic bipartite nuclear localization signal with the amino acid sequence of
(SEQ ID NO: 5)
PAAKRVKLDATESQDTGPPKKKRKV

16. The polynucleotide of any one of claims 7-12 wherein L₁, L₂ and L₃, as applicable, each, independently, comprises the amino acid sequence of (GGGGS)ₙ(SEQ ID NO: 22), (GGGSS)ₙ GPGSKLKSGFGGPGSRFRSGPG (SEQ ID NO: 1), or AAGGTGGGSGGGTGGS (SEQ ID NO: 2), or any combination thereof, wherein each n is, independently, 1-5.

17. The polynucleotide of any one of claims 7-12, wherein L₁, L₂ and L₃, as applicable, each, independently, comprises the amino acid sequence of MXGXG, IXGXG, LXGXG, MXGGX, IXGGX, LXGGX, wherein each X is any amino acid.

18. The polynucleotide of any one of claims 1-15, wherein the linker encoded by the polynucleotide is a peptide linker, including, but not limited to, GPGPG (SEQ ID NO: 32).

19. The polynucleotide of any one of claims 7-12, wherein L₂ comprises the amino acid sequence of: GPGSKLKSGFGGPGSRFRSGPG (SEQ ID NO: 1) or AAGGTGGGSGGGTGGS (SEQ ID NO: 2).

20. The polynucleotide of claim 9 or 10, wherein, as applicable, the C-terminus of bacteriophage repressor lambda comprises the amino acid sequence of:
(SEQ ID NO: 7)
LRSEYEYPVFSHVQAGMFSPELRTFTKGDAERWVSTTKKASDSAF
WLEVEGNSMTAPTGSKPSFPDGMLILVDPEQAVEPGDFCIARLGG
DEFTFKKLIRDSGQVFLQPLNPQYPMIPCNESCSVVGKVIASQWP
EETFG.

21. The polynucleotide of any one of claims 1-20, wherein, as applicable, the affinity binding domain is a heterologous tag.

22. The polynucleotide of claim 21, wherein the heterologous tag is a Flag tag, CBP tag, HA tag, HBH tag, Myc tag, histidine tag, S-tag, TAP, V5, or any combination thereof.

23. The polynucleotide of any one of claims 1-22, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 8, or comprises SEQ ID NO: 8.

24. The polynucleotide molecule of any one of claims 1-23, wherein the nucleotide sequence encoding for the CRO-NLS protein comprises a nucleic acid sequence having at least 85%, 86%, 87%, 88%, 89%, 90%, 91%, 92%, 93%, 94%, 95%, 96%, 97%, 98%, or 99% identity to SEQ ID NO: 9, or comprises the sequence of SEQ ID NO: 9.

25. The polynucleotide molecule of any one of claims 1-22, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 10, or comprises the sequence of SEQ ID NO: 10.

26. The polynucleotide molecule of any one of claim 1-22 or 25, wherein the sequence encoding for the CRO-NLS protein comprises a nucleic acid sequence having at least 85%, 86%, 87%, 88%, 89%, 90%, 91%, 92%, 93%, 94%, 95%, 96%, 97%, 98%, or 99% identity to SEQ ID NO: 11, or comprises the sequence of SEQ ID NO: 11.

27. The polynucleotide molecule of any one of claims 1-22, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 12, or comprises the sequence of SEQ ID NO: 12.

28. The polynucleotide molecule of any one of claim 1-22 or 27, wherein the sequence encoding for the CRO-NLS protein comprises a nucleic acid sequence having at least 85%, 86%, 87%, 88%, 89%, 90%, 91%, 92%, 93%, 94%, 95%, 96%, 97%, 98%, or 99% identity to SEQ ID NO: 13, or comprises the sequence of SEQ ID NO: 13.

29. The polynucleotide molecule of any one of claims 1-22, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 14, or comprises a sequence of SEQ ID NO: 14.

30. The polynucleotide molecule of any one of claims 1-22 or claim 29, wherein the sequence encoding for the CRO-NLS protein comprises a nucleic acid sequence having at least 85%, 86%, 87%, 88%, 89%, 90%, 91%, 92%, 93%, 94%, 95%, 96%, 97%, 98%, or 99% identity to SEQ ID NO: 15, or comprises the sequence of SEQ ID NO: 15.

31. The polynucleotide of any one of claims 1-30, wherein the at least one promoter region is upstream of the sequence encoding a CRO-NLS protein.

32. A polypeptide encoded by the polynucleotide of any one of claims 1-31.

33. A polypeptide, including an isolated polypeptide, comprising a CRO repressor protein operably connected to a nuclear localization signal (“CRO-NLS protein”).

34. The polypeptide of claim 33, the CRO repressor protein is linked to the NLS by a linker.

35. The polypeptide of claim 34, wherein the linker is a peptide linker, such as, but not limited to, a glycine/serine linker, including, but not limited to, (GGGGS)ₙ(SEQ ID NO: 22), (GGGSS)ₙ(SEQ ID NO: 23), (GSGSG)ₙ(SEQ ID NO: 24), (GSSG)ₙ(SEQ ID NO: 25), GPGSKLKSGFGGPGSRFRSGPG (SEQ ID NO: 1), or AAGGTGGGSGGGTGGS (SEQ ID NO: 2), or any combination thereof, wherein each n is, independently, 1-5.

36. The polypeptide of any one of claims 33-35, wherein the CRO-NLS comprises a monomer or dimer of a CRO repressor protein.

37. The polypeptide of any one of claims 33-36, wherein the CRO-NLS protein comprises a polypeptide having a formula of: X₂-L₂-X₃, wherein:
X₂ is a CRO repressor protein (e.g. monomer or dimer);
L₂ is a peptide linker, such as a flexible peptide linker, such as a glycine/serine linker; and
X₃ comprises at least one nuclear localization signal.

38. The polypeptide of any one of claims 33-36, wherein the CRO-NLS protein comprises a polypeptide having the formula of: X₁-L₁-X₂-L₂-X₃, wherein:
X₁ is an affinity binding domain;
X₂ is a CRO repressor protein;
X₃ comprises at least one nuclear localization signal or a synthetic bipartite nuclear localization signal; and
L₁ and L₂ are both, independently, flexible linker moieties, wherein L₁ and L₂ can be the same or different.

39. The polypeptide of any one of claims 33-36, wherein the CRO-NLS protein comprises a polypeptide having the formula of: X₂-L₂-X₃-L₃-X₄, wherein:
X₂ comprises the CRO protein,
X₃ comprises at least one nuclear localization signal or a synthetic bipartite nuclear localization signal,
X₄ comprises the C-terminus of bacteriophage repressor lambda, and
L₂ and L₃ are each, independently, flexible linker moieties, wherein L₂ and L₃ are the same or different.

40. The polypeptide of any one of claims 33-36, wherein the CRO-NLS protein comprises a polypeptide having the formula of: X₁-L₁-X₂-L₂-X₃-L₃-X₄, wherein:
X₁ comprises an affinity binding domain,
X₂ comprises the CRO protein,
X₃ comprises at least one nuclear localization signal or a synthetic bipartite nuclear localization signal,
X₄ comprises the C-terminus of bacteriophage repressor lambda, and
L₁, L₂, and L₃ are each, independently, flexible linker moieties, wherein L₁, L₂, and L₃ are the same or different.

41. The polypeptide of any one of claims 33-36, wherein the CRO-NLS protein comprises a polypeptide having the formula of: X₂-L₂-X₅-L₃-X₃, wherein:
X₂ comprises a first CRO protein;
X₅ comprises a second CRO protein;
X₃ comprises at least one nuclear localization signal; and
L₂, and L₃ are all, independently, flexible linker moieties, wherein L₁, L₂, and L₃ may comprise the same or unique linker sequences, and wherein L₂ may optionally be a non-cleavable linker.

42. The polypeptide of any one of claims 33-36, wherein the CRO-NLS protein comprises a polypeptide having the formula of: X₁-L₁-X₂-L₂-X₅-L₃-X₃, wherein:
X₁ comprises an affinity binding domain,
X₂ comprises a first CRO protein,
X₅ comprises a second CRO protein,
X₃ comprises at least one nuclear localization signal, and
L₁, L₂, and L₃ are all, independently, flexible linker moieties, wherein L₁, L₂, and L₃ may comprise the same or unique linker sequences, and wherein L₂ may optionally be a non-cleavable linker.

43. The polypeptide of claim 41 or 42, wherein the first CRO protein and the second CRO protein are the same or different.

44. The polypeptide of any one of claims 33-43, wherein the CRO protein comprises the amino acid sequence of:
(SEQ ID NO: 6)
MEQRITLKDYAMRFGQTKTAKDLGVYQSAINKAIHAGRKIFLTIN
ADGSVYAEEVKPFPSNKKTTA.

45. The polypeptide of any one of claims 33-44, wherein the NLS comprises the amino acid sequence of PAAKRVKLD (SEQ ID NO: 3); PKKKRKV (SEQ ID NO: 4); or PAAKRVKLDATESQDTGPPKKKRKV (SEQ ID NO: 5), or any combination thereof.

46. The polypeptide of any one of claims 33-45, wherein L₁, L₂, and L₃, as applicable, each, independently, comprises the amino acid sequence of (GGGGS)ₙ(SEQ ID NO: 22), (GGGSS)ₙ GPGSKLKSGFGGPGSRFRSGPG (SEQ ID NO: 1), or AAGGTGGGSGGGTGGS (SEQ ID NO: 2), or any combination thereof, wherein each n is, independently, 1-5.

47. The polypeptide of any one of claims 33-45, wherein L₁, L₂ and L₃, as applicable, each, independently, comprises the amino acid sequence of MXGXG, IXGXG, LXGXG, MXGGX, IXGGX, LXGGX, wherein each X is any amino acid.

48. The polypeptide of any one of claims 33-45, wherein L₁, L₂ and L₃, as applicable, each, independently, comprises an amino acid sequence of, including, but not limited to, GPGPG (SEQ ID NO: 32).

49. The polypeptide of claim 41 or 42, wherein L₂ comprises the amino acid sequence of: GPGSKLKSGFGGPGSRFRSGPG (SEQ ID NO: 1) or AAGGTGGGSGGGTGGS (SEQ ID NO: 2).

50. The polypeptide of claim 39 or 40, wherein the C-terminus of bacteriophage repressor lambda comprises the amino acid sequence of:
(SEQ ID NO: 7)
LRSEYEYPVFSHVQAGMFSPELRTFTKGDAERWVSTTKKASDSAF
WLEVEGNSMTAPTGSKPSFPDGMLILVDPEQAVEPGDFCIARLGG
DEFTFKKLIRDSGQVFLQPLNPQYPMIPCNESCSVVGKVIASQWP
EETFG.

51. The polypeptide of any one of claims 33-50, wherein, as applicable, the affinity binding domain is a heterologous tag.

52. The polypeptide of claim 51, wherein the heterologous tag is a Flag tag, CBP tag, HA tag, HBH tag, Myc tag, histidine tag, S-tag, TAP, V5, or any combination thereof.

53. The polypeptide of any one of claims 33-52, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 8, or comprises SEQ ID NO: 8.

54. The polypeptide of any one of claims 33-52, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 10, or comprises the sequence of SEQ ID NO: 10.

55. The polypeptide of any one of claims 33-52, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 12, or comprises the sequence of SEQ ID NO: 12.

56. The polynucleotide molecule of any one of claims 33-53, wherein the CRO-NLS protein comprises an amino acid sequence having at least 85%, 86%, 87%, 88%, 89%, 90%, 91%, 92%, 93%, 94%, 95%, 96%, 97%, 98%, or 99% identity to SEQ ID NO: 14, or comprises a sequence of SEQ ID NO: 14.

57. A cell comprising the polynucleotide of any one of claims 1-32 and a cargo polynucleotide comprising a nucleic acid sequence encoding for a molecule of interest, a promoter region operably connected to the molecule of interest, and at least a first CRO repressor binding site.

58. A cell comprising the polypeptide of any one of claims 33-56 and a cargo polynucleotide comprising a nucleic acid sequence encoding for a molecule of interest, a promoter region operably connected to the molecule of interest, and at least a first CRO repressor binding site.

59. The cell of claim 57 or 58, wherein the CRO repressor binding site is operably connected to the promoter region of the cargo polynucleotide, wherein in the presence of a CRO protein, the CRO protein inhibits the expression (e.g., transcription or translation) of the molecule of interest by binding to the CRO repressor binding site.

60. The cell of any one of claims 57-59, wherein the cargo polynucleotide comprises a nucleic acid sequence encoding for the molecule of interest, the promoter region operably connected to the molecule of interest, and the least a first CRO repressor binding site.

61. The cell of any one of claims 57-60, wherein the cargo polynucleotide comprises a 5′ adenoviral ITR and a 3′ adenoviral ITR, wherein the 5′ ITR and 3′ ITR flank the nucleic acid sequence encoding for the molecule of interest, the promoter region operably connected to the molecule of interest, and the least a first CRO repressor binding site.

62. The cell of any one of claims 57-61, wherein the promoter region of the cargo polynucleotide is located upstream of the sequence encoding the molecule of interest.

63. The cell of any one of claims 57-62, wherein the at least a first CRO repressor binding site is located upstream of the sequence encoding for the molecule of interest.

64. The cell of any one of claims 57-63, wherein the at least a first CRO repressor binding site is located upstream of the promoter region of the cargo polynucleotide.

65. The cell of any one of claims 57-63, wherein the at least a first CRO repressor binding site is located between the promoter region and the sequence encoding for the molecule of interest of the cargo polynucleotide.

66. The cell of any one of claims 57-63, wherein the at least a first CRO repressor binding site is located within the promoter region of the cargo polynucleotide.

67. The cell of any one of claims 57-66, wherein the at least a first CRO repressor binding site is selected from the group consisting of rightward operators of a bacteriophage and leftward operators of a bacteriophage.

68. The cell of any one of claims 57-67 wherein the at least a first CRO repressor binding site is selected from the group consisting of rightward operators of bacteriophage λ and leftward operators of bacteriophage 2.

69. The cell of any one of claims 57-68, wherein the at least a first CRO repressor binding site is selected from the group consisting of a bacteriophage λ leftward operator 1 (O_{L1}), a bacteriophage A leftward operator 2 (O_{L2}), a bacteriophage λ leftward operator 3 (O_{L3}), a bacteriophage λ rightward operator 1 (O_{R1}), a bacteriophage λ rightward operator 2 (O_{R2}), and a bacteriophage λ rightward operator 3 (O_{R3}).

70. The cell of any one of claims 57-69, wherein the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 16, or comprises a sequence of SEQ ID NO: 16.

71. The cell of any one of claims 57-69, wherein the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 17, or comprises a sequence of SEQ ID NO: 17.

72. The cell of any one of claims 57-69, wherein the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 18, or comprises a sequence of SEQ ID NO: 18.

73. The cell of any one of claims 57-69, wherein the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19, or comprises a sequence of SEQ ID NO: 19.

74. The cell of any one of claims 57-69, wherein the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 20, or comprises a sequence of SEQ ID NO: 20.

75. The cell of any one of claims 57-69, wherein the at least a first CRO repressor binding site comprises a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 21, or comprises a sequence of SEQ ID NO: 21.

76. The cell of any one of claims, 57-75, wherein the cargo polynucleotide comprises a second CRO repressor binding site.

77. The cell of claim 76, wherein the at least first and the second CRO repressor binding sites are upstream of the sequence encoding for the molecule of interest.

78. The cell of claim 76, wherein the at least first and the second CRO repressor binding sites are upstream of the promoter region.

79. The cell of claim 76, wherein the least first and the second CRO repressor binding sites are between the promoter region and the sequence encoding for the molecule of interest.

80. The cell of claim 76, wherein the at least first CRO repressor binding site is located within the promoter region and the second CRO repressor binding site is located between the promoter region and the sequence encoding for the molecule of interest.

81. The cell of claim 76, wherein the at least first CRO repressor binding site is located upstream of the promoter region and the second CRO repressor binding site is located within the promoter region.

82. The cell of any one of claims 76-81, wherein the second CRO repressor binding site is selected from the group consisting of rightward operators of a bacteriophage and leftward operators of a bacteriophage.

83. The cell of any one of claims 76-82, wherein the second CRO repressor binding site is selected from the group consisting of rightward operators of bacteriophage λ and leftward operators of bacteriophage 2.

84. The cell of any one of claims 76-83, wherein the second CRO repressor binding site is selected from the group consisting of a bacteriophage λ leftward operator 1 (O_{L1}), a bacteriophage λ leftward operator 2 (O_{L2}), a bacteriophage A leftward operator 3 (O_{L3}), a bacteriophage λ rightward operator 1 (O_{R1}), a bacteriophage λ rightward operator 2 (O_{R2}), and a bacteriophage λ rightward operator 3 (O_{R3}).

85. The cell of any one of claims 76-84, wherein the second CRO repressor binding site comprises:
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 16, or comprises a sequence of SEQ ID NO: 16;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 17, or comprises a sequence of SEQ ID NO: 17;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 18, or comprises a sequence of SEQ ID NO: 18;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19 or comprises a sequence of SEQ ID NO: 19;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 20 or a comprises a sequence of SEQ ID NO: 20; or
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 21 or a comprises a sequence of SEQ ID NO: 21.

86. The cell of any one of claims 57-85, wherein the sequence encoding the molecule of interest encodes for one or more of: a viral protein, a shRNA, therapeutic molecule, a tumor antigen, a protein, a nucleic acid molecule, or a combination thereof.

87. The cell of claim 86, wherein the therapeutic molecule is a cytokine, such as IL-2, IL-12, IL-15, and the like.

88. A virus comprising a cargo polynucleotide comprising a nucleic acid sequence encoding for a molecule of interest, a promoter region operably connected to the molecule of interest, and at least a first CRO repressor binding site.

89. The virus of claim 88, wherein the cargo polynucleotide comprises a 5′ adenoviral ITR and a 3′ adenoviral ITR, wherein the 5′ ITR and 3′ ITR flank the nucleic acid sequence encoding for the molecule of interest, the promoter region operably connected to the molecule of interest, and the least a first CRO repressor binding site.

90. The virus of claims 88 and 89, wherein the promoter region of the cargo polynucleotide is located upstream of the sequence encoding the molecule of interest.

91. The virus of any one of claims 88-90, wherein:
the at least a first CRO repressor binding site is located upstream of the sequence encoding for the molecule of interest;
the at least a first CRO repressor binding site is located upstream of the promoter region of the cargo polynucleotide;
the at least a first CRO repressor binding site is located between the promoter region and the sequence encoding for the molecule of interest of the cargo polynucleotide; or
the at least a first CRO repressor binding site is located within the promoter region of the cargo polynucleotide.

92. The virus of any one of claims 88-91, wherein the at least a first CRO repressor binding site is selected from the group consisting of rightward operators of a bacteriophage and leftward operators of a bacteriophage.

93. The virus of any one of claims 88-92, wherein the at least a first CRO repressor binding site is selected from the group consisting of rightward operators of bacteriophage λ and leftward operators of bacteriophage 2.

94. The virus of any one of claims 88-93, wherein the at least a first CRO repressor binding site is selected from the group consisting of a bacteriophage λ leftward operator 1 (O_{L1}), a bacteriophage A leftward operator 2 (O_{L2}), a bacteriophage A leftward operator 3 (O_{L3}), a bacteriophage λ rightward operator 1 (O_{R1}), a bacteriophage λ rightward operator 2 (O_{R2}), and a bacteriophage λ rightward operator 3 (O_{R3}).

95. The virus of any one of claims 88-94, wherein the at least a first CRO repressor binding site comprises:
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 16, or comprises a sequence of SEQ ID NO: 16;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 17, or comprises a sequence of SEQ ID NO: 17;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 18, or comprises a sequence of SEQ ID NO: 18;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19, or comprises a sequence of SEQ ID NO: 19;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 20, or comprises a sequence of SEQ ID NO: 20;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 21, or comprises a sequence of SEQ ID NO: 21.

96. The virus of any one of claims, 88-95, wherein the cargo polynucleotide comprises a second CRO repressor binding site.

97. The virus of claim 96, wherein:
the at least first and the second CRO repressor binding sites are upstream of the sequence encoding for the molecule of interest;
the at least first and the second CRO repressor binding sites are upstream of the promoter region;
the least first and the second CRO repressor binding sites are between the promoter region and the sequence encoding for the molecule of interest;
the at least first CRO repressor binding site is located within the promoter region and the second CRO repressor binding site is located between the promoter region and the sequence encoding for the molecule of interest; or
the at least first CRO repressor binding site is located upstream of the promoter region and the second CRO repressor binding site is located within the promoter region.

98. The virus of any one of claims 96-97, wherein the second CRO repressor binding site is selected from the group consisting of rightward operators of a bacteriophage and leftward operators of a bacteriophage.

99. The virus of any one of claims 96-98, wherein the second CRO repressor binding site is selected from the group consisting of rightward operators of bacteriophage λ and leftward operators of bacteriophage λ.

100. The virus of any one of claims 96-99, wherein the second CRO repressor binding site is selected from the group consisting of a bacteriophage A leftward operator 1 (O_{L1}), a bacteriophage λ leftward operator 2 (O_{L2}), a bacteriophage λ leftward operator 3 (O_{L3}), a bacteriophage λ rightward operator 1 (O_{R1}), a bacteriophage λ rightward operator 2 (O_{R2}), and a bacteriophage λ rightward operator 3 (O_{R3}).

101. The virus of any one of claims 96-100, wherein the second CRO repressor binding site comprises:
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 16, or comprises a sequence of SEQ ID NO: 16;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 17, or comprises a sequence of SEQ ID NO: 17;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 18, or comprises a sequence of SEQ ID NO: 18;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19 or comprises a sequence of SEQ ID NO: 19;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 20 or a comprises a sequence of SEQ ID NO: 20;
a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 21 or a comprises a sequence of SEQ ID NO: 21.

102. The virus of any one of claims 88-101, wherein the sequence encoding the molecule of interest encodes for one or more of: a viral protein, a shRNA, therapeutic molecule, a tumor antigen, a protein, a nucleic acid molecule, or a combination thereof.

103. The virus of claim 102, wherein the therapeutic molecule is a cytokine, such as IL-2, IL-12, IL-15, antigen, tumor antigen, protein, viral antigen, and the like.

104. The virus of claim 102, wherein the therapeutic molecule is any recombinant protein or RNA, including, but not limited, to a synthetic polytope.

105. A method of making a virus comprising the cargo polynucleotide, the method comprising culturing the cell of any one of claims 57-87 under conditions to produce the virus.

106. The method of claim 105, wherein the virus that is produced is an adenovirus.

107. The method of claim 105 or 106, wherein the virus is replication incompetent or replication competent.

108. A virus prepared according to a method of any one of claims 105-107.

109. A plasmid comprising the polynucleotide of any one of claims 1-31.

110. A cell comprising the polynucleotide of any one of claims 1-31 or the plasmid of claim 109.

111. A method of controlling expression of a molecule of interest, comprising:
contacting a host cell comprising a polynucleotide of any one of claims 1-31 or a polypeptide of any one of claims 32-56 with a cargo polynucleotide comprising a nucleic acid sequence encoding for a molecule of interest, a promoter region operably connected to the molecule of interest, and at least a first CRO repressor binding site,
wherein:
the expression of the molecule of interest is controlled by the binding of the polypeptide encoded for by the polynucleotide of any one of claims 1-31 or the polypeptide of any one of claims 32-56 to the CRO repressor binding site.

112. The method of claim 111, wherein the molecule of interest is one or more of: a viral protein, a shRNA, therapeutic molecule, a tumor antigen, a protein, a nucleic acid molecule, or a combination thereof.

113. A method of delivering a molecule of interest to a subject, the method comprising administering the virus of any one of claims 88-104 to the subject.

114. A method of inducing an immune response in a subject against a molecule of interest, the method comprising administering the virus of any one of claims 88-104 to the subject, wherein the molecule of interest is a viral protein or a tumor antigen.

115. A method of treating a disease, the method comprising administering the virus of any one of claims 88-104 to a subject to treat the disease.

116. The method of claim 115, wherein the disease is an infectious disease or a cancer.

117. A polypeptide comprising a nuclear localization signal (NLS) linked or fused to a heterologous molecule, wherein the NLS is a polypeptide having the formula of NLS₁-Xₙ-NLS₂, wherein NLS₁ and NLS₂ can comprise the same or different NLS sequences, Xₙ is a peptide linker, such as described herein.

118. The polypeptide of claim 117, wherein NLS₁ and NLS₂ are each, independently, selected from the group consisting of SEQ ID NO: 3, SEQ ID NO: 4, or a combination thereof.

119. The polypeptide of claim 117 or 118, wherein NLS₁ is SEQ ID NO: 3 and NLS₂ is SEQ ID NO: 4.

120. The polypeptide of any one of claims 117-119 wherein n is 9.

121. The polypeptide of any one of claims 117-120, wherein Xₙ comprises the amino acid sequence of ATESQDTGP (SEQ ID NO: 33).

122. The polypeptide of any one of claims 117-121, wherein the NLS comprises an amino acid sequence 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 5, or comprises a sequence of SEQ ID NO: 5.

123. The polypeptide of any one of claims 117-122, wherein the NLS comprises SEQ ID NO: 5.

124. The polypeptide of any one of claims 117-123, wherein the heterologous molecule is a polypeptide.

125. The polypeptide of any one of claims 117-123, wherein the heterologous molecule comprises a nucleic acid molecule.

126. The polypeptide of any one of claims 117-125, wherein the NLS and the heterologous molecule are joined or connected with a linker.

127. The polypeptide of claim 126, wherein the linker is a peptide linker, such as a peptide linker comprising the amino acid sequence of (GGGGS)ₙ(SEQ ID NO: 22), (GGGSS)ₙ(SEQ ID NO: 23), (GSGSG)ₙ(SEQ ID NO: 24), (GSSG)ₙ(SEQ ID NO: 25), GPGSKLKSGFGGPGSRFRSGPG (SEQ ID NO: 1), AAGGTGGGSGGGTGGS (SEQ ID NO: 2), MXGXG, IXGXG, LXGXG, MXGGX, IXGGX, LXGGX, GPGPG (SEQ ID NO: 32), or any combination thereof, wherein each n is, independently, 1-5 and wherein X is any amino acid.

128. The polypeptide of any one of claims 117-127, wherein the polypeptide further comprises a second heterologous molecule.

129. A polynucleotide molecule encoding the polypeptide of any one of claims 117-128.

130. The polynucleotide of claim 129, wherein the polynucleotide encoding the polypeptide is operably linked to a promoter.

131. A plasmid, cell, or virus comprising the polynucleotide molecule of claim 129 or 130.

132. A cell comprising the polypeptide of any one of claims 117-128.

133. A method of transporting a heterologous molecule of interest to the nucleus of a cell, the method comprising contacting the cell with a polypeptide of any one of claims 117-128.

134. A method for transporting a heterologous molecule of interest to the nucleus of a cell, the method comprising contacting the cell with a polynucleotide of claim 129 or 130 or with a plasmid comprising the same under conditions sufficient to express the molecule in the cell.

135. A method for transporting a heterologous molecule of interest to the nucleus of a cell, the method comprising contacting the cell with a vector comprising the polynucleotide of claim 129 or 130.


SEQUENCE LISTING (33 sequences)

SEQ ID NO: 1 - protein, 22 aa, synthetic construct
GPGSKLKSGFGGPGSRFRSGPG

SEQ ID NO: 2 - protein, 16 aa, synthetic construct
AAGGTGGGSGGGTGGS

SEQ ID NO: 3 - protein, 9 aa, synthetic construct
PAAKRVKLD

SEQ ID NO: 4 - protein, 7 aa, synthetic construct
PKKKRKV

SEQ ID NO: 5 - protein, 25 aa, synthetic construct
PAAKRVKLDATESQDTGPPKKKRKV

SEQ ID NO: 6 - protein, 66 aa, synthetic construct
MEQRITLKDYAMRFGQTKTAKDLGVYQSAINKAIHAGRKIFLTINADGSVYAEEVKPFPSNKKTTA

SEQ ID NO: 7 - protein, 140 aa, synthetic construct
LRSEYEYPVFSHVQAGMFSPELRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 8 - protein, 108 aa, synthetic construct
MDYKDHDGDYKDHDIDYKDDDDKGGGSSMEQRITLKDYAMRFGQTKTAKDLGVYQSAINKAIHAGRKIFLTINADGSVYAEEVKPFPSNKKTTAGSGSGPAAKRVKLD

SEQ ID NO: 9 - protein, 327 aa, synthetic construct
ATGGATTACAAGGACCACGACGGTGACTATAAAGACCATGATATTGATTACAAAGATCATGACGACAAGGGCGGGGGAAGTAGTATGGAACAGCGGATAACTTTGAAAGACTATGCAATGCGCTTCGGCCAAACCAAGACTGCTAAAGACTTGGGTGTGTACCAAAGTGCAATAAATAAAGCTATCCACGCCGGGAGGAAGATCTTTCTCACGATAAACGCTGACGGCTCCGTGTATGCCGAAGAAGTGAAACCTTTCCCTTCTAATAAAAAGACCACTGCGGGTTCCGGCAGTGGCCCGGCTGCAAAGCGGGTCAAACTTGATTAA

SEQ ID NO: 10 - protein, 268 aa, synthetic construct
MDYKDHDGDYKDHDIDYKDDDDKGGGSSMEQRITLKDYAMRFGQTKTAKDLGVYQSAINKAIHAGRKIFLTINADGSVYAEEVKPFPSNKKTTAGSGSGPAAKRVKLDATESQDTGPPKKKRKVGSSGLRSEYEYPVFSHVQAGMFSPELRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 11 - protein, 807 aa, synthetic construct
ATGGACTACAAGGACCATGATGGTGATTATAAAGACCACGATATAGACTACAAAGACGACGATGATAAGGGCGGGGGAAGTAGTATGGAACAGCGCATAACGTTGAAAGACTACGCAATGCGATTCGGTCAAACAAAGACGGCTAAAGACTTGGGTGTTTACCAGAGTGCGATAAACAAGGCCATCCATGCGGGAAGAAAGATATTTTTGACGATAAATGCAGATGGCTCCGTTTACGCGGAGGAAGTCAAGCCCTTTCCCTCAAATAAAAAAACAACGGCTGGTTCCGGCAGTGGCCCTGCCGCCAAACGGGTGAAACTGGACGCGACAGAGTCACAGGACACCGGCCCACCAAAGAAAAAAAGAAAGGTGGGCAGTTCCGGGCTCCGAAGCGAATACGAGTACCCGGTGTTCTCCCATGTACAGGCCGGAATGTTTTCTCCGGAACTCAGAACCTTCACGAAAGGCGATGCGGAGAGGTGGGTGAGTACCACCAAAAAGGCCTCTGACAGTGCTTTTTGGCTTGAAGTAGAAGGGAACTCCATGACTGCGCCGACCGGATCTAAACCGAGTTTCCCGGATGGAATGCTCATCCTGGTCGATCCTGAACAAGCTGTTGAACCGGGTGATTTCTGTATCGCCCGCCTGGGGGGTGACGAGTTCACCTTCAAAAAGCTCATTCGGGACTCCGGACAAGTTTTTTTGCAACCGCTTAATCCCCAGTACCCTATGATTCCCTGCAATGAGTCCTGCTCTGTAGTGGGTAAAGTGATAGCTAGTCAGTGGCCGGAAGAAACATTCGGGTAA

SEQ ID NO: 12 - protein, 190 aa, synthetic construct
MDYKDHDGDYKDHDIDYKDDDDKGGGSSMEQRITLKDYAMRFGQTKTAKDLGVYQSAINKAIHAGRKIFLTINADGSVYAEEVKPFPSNKKTTAAAGGTGGGSGGGTGGSMEQRITLKDYAMRFGQTKTAKDLGVYQSAINKAIHAGRKIFLTINADGSVYAEEVKPFPSNKKTTAGSGSGPAAKRVKLD

SEQ ID NO: 13 - protein, 573 aa, synthetic construct
ATGGACTACAAAGACCATGATGGGGACTATAAGGATCACGACATAGACTATAAGGACGATGATGACAAGGGCGGAGGAAGCAGCATGGAACAGAGAATCACACTCAAAGACTATGCAATGAGATTCGGTCAGACGAAAACTGCCAAGGATCTCGGCGTCTACCAAAGCGCGATCAACAAAGCAATTCATGCGGGACGAAAAATTTTTCTGACGATAAATGCTGATGGATCAGTGTACGCAGAAGAGGTAAAGCCTTTCCCGTCAAACAAGAAAACGACAGCGGCAGCGGGCGGCACGGGGGGAGGTTCCGGGGGCGGGACAGGTGGCTCTATGGAGCAACGCATTACTCTGAAGGACTACGCCATGAGATTCGGGCAGACAAAGACTGCTAAGGATTTGGGGGTATATCAATCAGCAATCAATAAGGCAATACATGCAGGGCGCAAAATTTTTCTTACGATAAACGCTGATGGAAGTGTATACGCTGAAGAAGTAAAACCTTTCCCAAGTAATAAGAAAACGACCGCCGGTAGCGGGAGCGGACCAGCCGCCAAGCGGGTAAAACTCGATTAA

SEQ ID NO: 14 - protein, 196 aa, synthetic construct
MDYKDHDGDYKDHDIDYKDDDDKGGGSSMEQRITLKDYAMRFGQTKTAKDLGVYQSAINKAIHAGRKIFLTINADGSVYAEEVKPFPSNKKTTAGPGSKLKSGFGGPGSRFRSGPGMEQRITLKDYAMRFGQTKTAKDLGVYQSAINKAIHAGRKIFLTINADGSVYAEEVKPFPSNKKTTAGSGSGPAAKRVKLD

SEQ ID NO: 15 - protein, 591 aa, synthetic construct
ATGGACTACAAAGACCATGATGGGGACTATAAGGATCACGACATAGACTATAAGGACGATGATGACAAGGGCGGAGGAAGCAGCATGGAACAGAGAATCACACTCAAAGACTATGCAATGAGATTCGGTCAGACGAAAACTGCCAAGGATCTCGGCGTCTACCAAAGCGCGATCAACAAAGCAATTCATGCGGGACGAAAAATTTTTCTGACGATAAATGCTGATGGATCAGTGTACGCAGAAGAGGTAAAGCCTTTCCCGTCAAACAAGAAAACGACAGCGGGACCTGGAAGCAAACTTAAAAGCGGATTTGGAGGGCCCGGGTCACGCTTCCGCTCTGGACCGGGCATGGAGCAACGCATTACTCTGAAGGACTACGCCATGAGATTCGGGCAGACAAAGACTGCTAAGGATTTGGGGGTATATCAATCAGCAATCAATAAGGCAATACATGCAGGGCGCAAAATTTTTCTTACGATAAACGCTGATGGAAGTGTATACGCTGAAGAAGTAAAACCTTTCCCAAGTAATAAGAAAACGACCGCCGGTAGCGGGAGCGGACCAGCCGCCAAGCGGGTAAAACTCGATTAA

SEQ ID NO: 16 - protein, 18 aa, synthetic construct
TATCACCGCCAGTGGTAT

SEQ ID NO: 17 - protein, 19 aa, synthetic construct
TCAACACCGCCAGAGATAA

SEQ ID NO: 18 - protein, 20 aa, synthetic construct
TTATCACCGCAGATGGTTAT

SEQ ID NO: 19 - protein, 19 aa, synthetic construct
TTATCACCGCCAGAGGTAA

SEQ ID NO: 20 - protein, 19 aa, synthetic construct
TCAACACGCACGGTGTTAG

SEQ ID NO: 21 - protein, 19 aa, synthetic construct
TTATCCCTTGCGGTGATAG

SEQ ID NO: 22 - protein, 5 aa, synthetic construct
GGGGS

SEQ ID NO: 23 - protein, 5 aa, synthetic construct
GGGSS

SEQ ID NO: 24 - protein, 5 aa, synthetic construct
GSGSG

SEQ ID NO: 25 - protein, 4 aa, synthetic construct
GSSG

SEQ ID NO: 26-31 - skipped (6 entries)

SEQ ID NO: 32 - protein, 5 aa, synthetic construct
GPGPG

SEQ ID NO: 33 - protein, 9 aa, synthetic construct
ATESQDTGP